(19) United States
(12) Patent Application Publication
(10) Pub. No.: US 2021/0380960 A1
(43) Pub. Date: Dec. 9, 2021

(54) USE OF A COMBINATION OF TET EXOPROTEASES OBTAINED FROM EXTREMOPHILIC MICROORGANISMS FOR HYDROLYZING POLYPEPTIDES

(71) Applicants: Centre national de la recherche scientifique, PARIS (FR); Commissariat à l'énergie atomique et aux énergies alternatives, PARIS (FR); UNIVERSITE GRENOBLE ALPES, SAINT MARTIN D'HERES (FR)

(72) Inventors: Bruno FRANZETTI, SASSENAGE (FR); Eric GIRARD, ROMANS-SUR-ISERE (FR); Alexandre APPOLAIRE, GRENOBLE (FR)

(21) Appl. No.: 16/771,794

(22) PCT Filed: Dec. 12, 2018
(86) PCT No.: PCT/EP2018/084532

§ 371 Date: Feb. 1, 2021

(30) Foreign Application Priority Data
Dec. 12, 2017 (EP) 17306755.4

(51) Int. Cl. C12N 9/48; C12N 9/52; C12P 21/06; A23L 33/18; A23L 33/185; A23L 33/19; A23C 21/02; A23J 3/34; A23L 5/20

(52) U.S. Cl. CPC C12N 9/485; C12N 9/52; C12Y 304/24027; C12P 21/06; C12Y 304/11; A23L 33/18; A23V 2002/00; A23L 33/19; A23C 21/02; A23J 3/344; A23J 3/346; A23L 5/25; A23L 33/185

(57) ABSTRACT

The invention relates to a composition comprising at least one first aminopeptidase and at least one second aminopeptidase, the first aminopeptidase representing up to 40% by weight relative to the total weight of the composition.

The present invention relates to a composition comprising aminopeptidases, more particularly a composition comprising tetrahedral aminopeptidases, also called “TET aminopeptidases”.

In the food industry, “long” peptides, also known as “complexes”, are naturally present in the food to be consumed. These long peptides are generally the result of an insufficient degradation process and are sometimes toxic, which causes intolerance or allergies to foods containing these peptides, and also decreases their nutritional efficiency. These peptides also determine the taste and texture of many foods such as breads, cheeses, cured meats, etc.

Gluten intolerance is probably one of the most common food intolerances. It is caused by a family of complex proteins called “gliadins”, which enter into the composition of gluten, wherein these proteins sometimes carry a peptide called “immunodominant”, which causes an allergic reaction in people sensitive or intolerant to gluten.

For these reasons, the food industry uses proteolytic enzymes in many processes aimed at degrading gluten and making it less immunogenic. These are essentially endoproteases which make it possible to cleave proteins and long peptides into fragments. These endoproteases are used, in particular, in the production processes of pastry, cheese, biscuits, but also in the production of fruit juice or beer, and even in the production of protein hydrolyzate intended for special food. These proteases generally come from bacteria or fungi and their exact nature is generally kept confidential.

However, a disadvantage of the proteases currently used is that they do not allow the production of peptides that are short enough to eliminate all the toxic parts of food peptides, or that enable the peptides to be efficiently digested. In people sensitive or intolerant to gluten, for example, the incomplete digestion of gluten induces the presence of the “immunodominant” peptide in the digestive system and ultimately causes the symptoms of Celiac disease.

Mesophilic aminopeptidases have already been developed for the degradation of these complex food peptides, but their weak catalytic activity and the physicochemical conditions under which they are active, limit their use solely to the modification of the taste of the food.

Asunción Dura et al. (The structural and biochemical characterizations of a novel TET peptidase complex from Pyrococcus horikoshii reveal an integrated peptide degradation system in hyperthermophilic Archaea, Molecular Microbiology (2009) 72 (1), 26-40), which relates to Pyrococcus horikoshii expressing three aminopeptidases, characterizes the peptidase PhTET3 and compare it to PhTET1 and PhTET2. These are not compositions comprising aminopeptidases derived from extremophilic microorganisms, i.e. compositions comprising aminopeptidases isolated from extremophilic microorganisms, and, therefore, they are very different from the original microorganism itself.

The invention aims to remedy all these drawbacks. The invention, therefore, relates to a composition comprising at least one first aminopeptidase and at least one second aminopeptidase, said first and second aminopeptidases being different from each other, said first and second aminopeptidases being derived from extremophilic microorganisms, said first and second aminopeptidases being aminopeptidases of the family of tetrahedral aminopeptidases or TET aminopeptidases, said first aminopeptidase representing up to 40% by weight relative to the total weight of the composition, wherein, if said first and second aminopeptidases are different from PhTET2 and PhTET3, then said first aminopeptidase represents up to 50% by weight relative to the total weight of the composition. The inventors made the surprising observation that the use of a composition comprising at least two TET aminopeptidases is capable of degrading peptides, especially in admixture, and exerting a synergistic effect going beyond the additive effect of individual activities of each of the TET proteins. In fact, instead of observing an overall activity of the composition corresponding to the combination of the activities of the various TET aminopeptidases present in the composition, the inventors found a different overall activity which may be modulated by the physicochemical conditions of the reaction medium or by interactions/interferences between the different TET aminopeptidases of the composition.

By “aminopeptidase” is meant an enzyme exhibiting amino acid cleavage activity at the end of peptides, polypeptides or proteins. By “peptide” according to the invention is meant a chain of amino acids comprising at least 2 amino acids. The peptides may be obtained either from the degradation of proteins, or from chemical syntheses. By “polypeptide” according to the invention is meant a chain of amino acids larger than a chain of amino acids of a peptide, and that is obtained from the degradation of proteins and not from chemical synthesis. Peptides and polypeptides may have a biological function. However, peptides and polypeptides cannot exercise this function alone as part of a cellular process. By “protein” according to the invention is meant a molecule containing a chain of amino acids having a biological function and which is found naturally in an organism. This biological function is part of a natural process in the cell. The composition therefore comprises two aminopeptidases different from each other, wherein each has an amino acid sequence different from each other. In other words, the aminopeptidases have amino acid sequences which diverge by one amino acid. In other words, the sequences of the two aminopeptidases diverge from each other by one or more amino acids.

The tetrahedral aminopeptidases or TET aminopeptidases used in the composition of the invention are isolated from extremophilic microorganisms and, more particularly, from marine extremophilic microorganisms. These tetrahedral aminopeptidases or TET aminopeptidases, belong to the metalloaminopeptidase families M42 and M18 according to the MEROPS classification. In other words, the TET aminopeptidases are in the form of enzyme complexes comprising 12 subunits which have the particularity of self-assembling into constructions forming typical tetrahedron structures. Such a form contributes to the great stability of the enzyme. By “metallo aminopeptidases” is meant that these aminopeptidases all have in common the presence of at least one metal ion within the active site.

By “extremophilic microorganisms” is meant living organisms, invisible to the naked eye, which may only be observed using a microscope. These microorganisms may take various forms of life including bacteria, microscopic fungi, archaeabacteria, protists, microscopic green algae, plankton animals, planaria, amoebae and viruses. “Extremophile” describes an organism whose normal living conditions generally represent fatal conditions for most of the other organisms. These living conditions may be high or low temperature, extreme pressures, high salinity, acidity or alkalinity of the environment in which the organism lives, the presence of radioactivity or the absence of oxygen or light.

These TET aminopeptidases differ from other aminopeptidases in that they may be very specific for certain types of amino acids. Consequently, it is possible with the compositions according to the invention, to combine different TET aminopeptidases, so as to degrade peptides or so-called complex proteins into sufficiently short peptides. Therefore, it is possible to degrade all the toxic parts, so that they may be effectively digested. Such a final result is obtained thanks to an aminopeptidase activity specific to the composition used. This aminopeptidase activity is established with regard to the polypeptide content of the substrate, and of the parts which it is desired to modify, or even completely degrade. To do this, we choose for the composition of the invention, the associated TET aminopeptidases as a function of their specificity for certain amino acids and of their interaction/interference with each other with regard to their activity. In addition, it has been found by the inventors that certain TET aminopeptidases exhibit, under certain conditions, better activity for the degradation of peptides enriched in a particular amino acid type than other TET aminopeptidases of which it is known in the prior art, that they are specific for this type of amino acid. In other words, the various associations of TET aminopeptidases are not a simple addition of the known activities of the TET aminopeptidases which are associated in the composition.

In the composition according to the invention, the first aminopeptidase may represent up to 40% by weight relative to the total weight of the composition. By “up to 40% relative to the total weight of the composition” is meant that the first aminopeptidase may represent 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% relative to the total weight of the composition. When a composition consists only of two TET aminopeptidases, the second aminopeptidase may therefore represent at least 60% by weight relative to the total weight of the composition, which means 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Then, the first and second TET aminopeptidases may be in proportions ranging from 1:99 by weight relative to the total weight of the composition up to 40:60 by weight relative to the total weight of the composition, as a function of the weight of the first aminopeptidase relative to the total weight of the composition.

However, if the first aminopeptidase and the second aminopeptidase are not PhTET2 and PhTET3, then the first aminopeptidase may represent up to 50% by weight relative to the total weight of the composition. By “up to 50% relative to the total weight of the composition” according the invention is meant that the first aminopeptidase may represent 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50% relative to the total weight of the composition.

By “said first and second aminopeptidases are different from PhTET2 and PhTET3” is meant that said first and second aminopeptidases comprise, consist essentially of, or consist of amino acid molecules whose sequences have less than 65% identity with the sequence PhTET2 (SEQ ID NO: 2) or the sequence PhTET3 (SEQ ID NO: 3). By “% identity between two sequences” is meant that when these two amino acid sequences are aligned in order to compare them, by any means known to those skilled in the art, these two sequences present portions of sequence whose amino acid chains are identical. All of these portions establish the percentage of identity between the two sequences. “By less than 65% identity with the PhTET2 sequence (SEQ ID NO: 2) or the PhTET3 sequence (SEQ ID NO: 3)” is meant: 1%, 2%, 3%, 4%, 5% 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63% or 64% identity with the PhTET2 sequence (SEQ ID NO: 2) or the PhTET3 sequence (SEQ ID NO: 3).

This means that in the embodiment where the composition consists only of the first and second TET aminopeptidases, the second TET aminopeptidase nay represent at least 50% by weight relative to the total weight of the composition, which means 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, if the first aminopeptidase and the second aminopeptidase are not PhTET2 and PhTET3.

Advantageously, said at least one first aminopeptidase and said at least one second aminopeptidase are chosen from aminopeptidases from the group consisting of: PhTET1, PhTET2, PhTET3, PhTET4 and MjTET.

PhTET1 is represented by the amino acid sequence SEQ ID NO: 1, PhTET2 by the amino acid sequence SEQ ID NO: 2., PhTET3 by the amino acid sequence SEQ ID NO: 3, PhTET4 by the amino acid sequence SEQ ID NO: 4, and MjTET by the amino acid sequence SEQ ID NO: 5.

In other words, the pairs of first and second TET aminopeptidases of a composition according to the invention may be chosen from the list of couples consisting of: PhTET1-PhTET2, PhTET1-PhTET3, PhTET1-PhTET4, PhTET1-MjTET, PhTET2-PhTET3, PhTET2-PhTET4, PhTET2-MjTET, PhTET3-PhTET4, PhTET3-MjTET, PhTET4-MjTET.

Advantageously, said aminopeptidases comprise, consist essentially of, or consist of, amino acid molecules of respective sequences SEQ ID NO: 1 to SEQ ID NO: 5, or proteins exhibiting aminopeptidase activity, said proteins comprising, consisting essentially of, or consisting of, amino acid molecules, whose sequences have at least 65% identity with one of the sequences SEQ ID NO: 1 to SEQ ID NO: 5.

By “% identity with one of the sequences SEQ ID NO: 1 to SEQ ID NO: 5” according to the invention is meant that the proteins have an amino acid sequence which, when it aligns with one of the sequences SEQ ID NO: 1 (sequence of PhTET1), SEQ ID NO: 2 (sequence of PhTET2), SEQ ID NO: 3 (sequence of PhTET3), SEQ ID NO: 4 (sequence of PhTET4) or SEQ ID NO: 5 (MjTET sequence) so as to compare the two sequences, makes it possible to observe portions of sequence whose amino acid sequences are identical from one sequence to another. By “at least 65% identity with one of the sequences SEQ ID NO: 1 to SEQ ID NO: 5” according to the invention is meant that the percentage of identity may be: 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% of identity. In other words, the proteins considered are those having an amino acid sequence whose percentage of identity with one of the sequences SEQ ID NO: 1 to SEQ ID NO: 5 is one of those described above and having a aminopeptidase activity, i.e. an amino acid cleavage activity at the N-terminal end of the polymers.

Structural studies clearly show that even with a low level of homology, enzymes may be assigned to the TET family on the basis of specific criteria. However, while the various enzyme complexes show great structural homology, the percentage of sequence identity between the TETs is not very high. For example, PhTET1 shows approximately 37% identity (55% homology) with PhTET2 and PhTET3, while the latter two show 48% identity (70% homology) between them.

As in the case of the reference to the thesis of Dr. Alexandre APPOLAIRE on “Study of the large proteolytic assemblies of the TET family: oligomerization process and associated functional regulation.”, Incorporated here by reference, the inventors retained 5 criteria to characterize a protein exhibiting aminopeptidase activity:

1) The insertion of the dimerization domain into the sequence: this insertion of a specific domain in the middle of the gene coding for aminopeptidase is a first strong marker of the structure of TET aminopeptidases (Schoehn, G., Vellieux, F. M. D., Asunción Durá, M., Receveur-Bréchot, V., Fabry, C. M. S., Ruigrok, R. W. H., Ebel, C., Roussel, A., and Franzetti, B., (2006) An archaeal peptidase assembles into two different quaternary structures: A tetrahedron and a giant octahedron The Journal of biological chemistry. 281: 36327-36337).

2) The dimerization interface: The presence of the “IDIGAXXXE” pattern appears to be decisive for the assembly of TET dimers. The presence of R320Y321 residues also appears to be important.

3) The dodecamerization interface: this is an area where the interactions are less well defined; it involves polar, hydrophobic interactions, hydrogen bonds and a salt bridge. The presence of the α5 helix at the interface between the monomers in an adequate conformation associated with the presence of residues with a long side chain loaded on it, appears to be decisive. However, it seems difficult to determine a standard sequence that could be identified in the genomes.

4) Folding of the catalytic domain: the presence of several very conserved glycines seems to be important for the folding of the catalytic domain of TETs.

5) The active site: The conservation of the site is part of the characteristics of a TET and of the peptidases of the family M42, it is defined by the following residues: H65XD67, D181, E213E214, E/D236 and H319 in PhTET3, and involves the coordination of two metal ions. These ions are, in general, CO²⁺ or Zn²⁺ ions, but certain structures have been characterized with different ions; the structure of Q11Z05_CYTH3 has, for example, been characterized with active sites carrying Fe²⁺ ions.

According to the inventors, these five criteria must be met so as to identify a peptidase as a tetrahedral TET peptidase.

Advantageously, said first aminopeptidase represents up to 10% by weight relative to the total weight of the composition, in particular up to 5% by weight relative to the total weight of the composition.

Thus, it is possible to very precisely modulate the actions of the aminopeptidases of the composition with the addition of a first aminopeptidase in small quantities. Consequently, this addition of a TET aminopeptidase in small amounts to the composition, makes it possible to have a significant influence on the nature of the products obtained after bringing the polypeptide content of a substrate into contact with the composition of the invention.

Advantageously, said first aminopeptidase represents 50% by weight relative to the total weight of the composition, provided that said first and second aminopeptidases are different from PhTET2 and PhTET3.

In other words, in the alternative of the composition according to the invention, where the latter consists only of the first aminopeptidase and the second aminopeptidase, these two TET aminopeptidases may be in equimolar proportions, provided that said first and second aminopeptidases are different from PhTET2 and PhTET3.

Advantageously, the composition of the invention comprises at least a third aminopeptidase, said third aminopeptidase being an aminopeptidase from the family of tetrahedral aminopeptidases or TET aminopeptidases. In the case where the composition comprises three TET aminopeptidases, the triplets may be chosen from the list of triplets consisting of: PhTET1-PhTET2-PhTET3, PhTET1-PhTET2-PhTET4, PhTET1-PhTET2-MjTET, PhTET1-PhTET3-PhTET4, PhTET1-PhTET3-MjTET, PhTET1-PhTET4-MjTET, PhTET2-PhTET3-PhTET4, PhTET2-PhTET3-MjTET, PhTET2-PhTET4-MjTET, PhTET3-PhTET4-MjTET. Furthermore, in the case where the composition comprises four TET aminopeptidases, the quadruplets may be chosen from the list of quadruplets consisting of: PhTET1-PhTET2-PhTET3-PhTET4, PhTET1-PhTET2-PhTET3-MjTET, PhTET2-PhTET3-PhTET-MjTE, PhTET1-PhTET3-PhTET4-MjTET. Furthermore, in the case where the composition contains five aminopeptidases, the quintuplet may be PhTET1-PhTET2-PhTET3-PhTET4-MjTET.

Thus, it is possible to add the action of a third TET aminopeptidase, different from the first TET aminopeptidase and the second TET aminopeptidase, which thus makes it possible to improve the field of action of the composition and thus modify the targeted food peptides, polypeptides and proteins even better. Consequently, the first TET aminopeptidase may represent up to 40% by weight relative to the total weight of the composition, while the at least 60% by weight relative to the total weight of the remaining composition may be distributed between the second TIN aminopeptidase and the third TET aminopeptidase.

However, when the first and second aminopeptidases are different from PhTET2 and PhTET3, the composition may be such in proportions that the first TET aminopeptidase represents up to 50% by weight relative to the total weight of the composition, while the at least 50% by weight relative to the total weight of the remaining composition may be divided between the second TET aminopeptidase and the third TET aminopeptidase.

By way of examples, there are, therefore, embodiments according to which the composition consists of a first TET aminopeptidase, a second TET aminopeptidase, and a third TET aminopeptidase, and, according to which, the proportions by weight relative to the total weight of the composition of the first, second and third aminopeptidases are the following: 50/25/25, 40/30/30, 40/40/20, 10/10/80 or even 10/20/70.

Advantageously, said first, second and third aminopeptidases are in equimolar or substantially equimolar proportions.

By “equimolar or substantially equimolar proportions” is meant that the quantities of the first, second and third aminopeptidases are the same or substantially the same, i.e. they are very close to one another. Such precision makes it possible to precisely define the aminopeptidase activity of a composition, and, thus, to better adapt to the peptide to be degraded.

Advantageously, the composition also comprises an endopeptidase, in particular thermolysin, in particular thermolysin of sequence SEQ ID NO: 6. Thermolysin is an endopeptidase belonging to the family of metalloproteinases. Thermolysin is the most stable member of a family of metalloproteinases produced by various Bacillus species. Unlike many proteins that undergo conformational changes during heating and denaturation, thermolysin undergoes no major conformational changes up to at least 70° C. Thermolysin therefore remains stable and active at temperatures where the TET proteins are most active.

“Endopeptidase” means an enzyme capable of breaking the bonds between non-terminal amino acids of a peptide, polypeptide or protein.

Thus, the composition exhibits a broader aminopeptidase activity thanks to the possibility of cleaving non-terminal amino acids.

The invention also relates to a use of a composition according to the invention for the modification of all or part of the polypeptide content of a substrate comprising peptides, polypeptides and/or proteins.

“Modification of all or part of the polypeptide content of a substrate” means at least one modification of part of the polypeptide content of a substrate. In other words, the polypeptide content of the substrate is different from the polypeptide content of the product obtained after being brought into contact with the composition according to the invention. For example, in the case of a content of polypeptides comprising three proteins, the modification of all the content corresponds to the complete degradation of the three proteins. Conversely, the modification of part of this content may correspond to the complete degradation of one protein of the three, or to the partial modification of one protein, of two proteins of the three, or even of the three proteins of the substrate. Those skilled in the art are able to determine whether a protein content is fully or partially modified.

In the invention, no distinction is made between the expressions “modification of all or part of the polypeptide content of a substrate” and “degradation of all or part of the polypeptide content of a substrate”. These expressions may replace one another without problem with regard to the subject matter of the invention. In fact, a modification of a content of polypeptides is the result of a degradation of at least one of the constituents (peptide, polypeptide or protein) of this content.

Thus, the composition according to the invention may be used to modify all or part of the polypeptide content of a substrate which comprises peptides, polypeptides and/or proteins. These peptides, polypeptides and/or proteins undergo the action of the TET aminopeptidases forming all or part of the composition, and are thus modified into short peptides, which allows the elimination of their toxic part and better digestion of the peptide content of the product obtained after bringing the polypeptide content of the substrate into contact with the composition.

Advantageously, the substrate comprises at least peptides, polypeptides and/or proteins of the gluten and/or of the whey.

Thus, it is possible by using the composition according to the invention, to modify the peptides, polypeptides and/or proteins of gluten or whey, wherein these two protein assemblies comprise peptides, polypeptides and/or proteins, which are the cause of food intolerances and allergies for end consumers. As mentioned earlier, gluten mainly consists of two families of proteins: gliadins and glutenins. These proteins are insoluble in water and give the dough, obtained after rehydration of the flour, viscoelastic properties used in the food industry to give a certain structure to the products. Whey contains most of the milk water. It consists of 94% water, 4 to 5% lactose, soluble proteins (9% dry matter), and mineral salts. Whey proteins are of real nutritional value due to their high composition of essential amino acids. The most important are ß-lactoglobulin (ß-LG), α-lactalbumin (α-LA), bovine immunoglobulins (IgG), bovine serum albumin (BSA) and bovine lactoferrin (LF).

Advantageously, the substrate comprises at least one of the following proteins: gliadin, ß-lactoglobulin, α-lactalbumin, immunoglobulins, serum albumin, and lactoferrin.

As mentioned above, the use of the composition according to the invention allows the modification of these proteins which are the cause of food intolerances and allergies among end consumers.

Advantageously, said aminopeptidases forming all or part of the composition may be used simultaneously, separately or spread over time.

This possibility of using these aminopeptidases simultaneously, separately or spread over time, makes it possible to adapt the overall activity of the composition to the polypeptide content of the substrate on which the composition is used. In fact, it is possible to have recourse to the different aminopeptidase activities of the composition, functions inter alia of the aminopeptidases, of their proportion, and of their interaction with each other in terms of activity, in order to adapt the action of modifying the content of substrate polypeptides, over time. The aminopeptidases forming the composition may be used simultaneously. As such, they may, for example, be added at the same time in a medium comprising the substrate to be modified. Aminopeptidases may also be used separately. For example, in the case of a large chain, part of the aminopeptidases may be added to one end of the chain, while another part is added to another end of the chain. Such a solution may be developed with the aim of obtaining a homogeneous distribution within the chain of added aminopeptidases more quickly. This solution may also be developed when an aminopeptidase introduced at one end of the chain requires a certain period of adaptation within the medium before being suitable to for bring into contact with other aminopeptidases added at another end of the chain. Aminopeptidases may also be used over time. Thus, it is possible to add an aminopeptidase, for a certain time, in order to modify the polypeptide content of the substrate. Therefore, it is possible to add one or more other aminopeptidases, so as to modify all or part of the polypeptide content resulting from the modification by the first aminopeptidase, taking into account the interactions/interferences of the first aminopeptidase already present in the reaction medium, on the activity of this new added aminopeptidase.

In an advantageous aspect of the invention, the TET proteins may be fixed on a support, in particular a column, silica or also magnetic beads, or any other support suitable for fixing the proteins.

In particular, the enzymes may be used in the form of crosslinked enzyme crystals or CLEC (Cross-Linked Enzyme Crystals).

The invention therefore relates to a composition in which one or more aminopeptidases, preferably all of the aminopeptidases of the composition, are in the form of crosslinked crystals.

The invention thus relates to a composition in which one or more aminopeptidases, preferably all of the aminopeptidases of the composition, are immobilized, in particular in the form of crosslinked crystals.

The invention also relates to a method for modifying all or part of the polypeptide content of a substrate comprising peptides, polypeptides and/or proteins, said method comprising a contacting step:
said substrate with
a composition according to the above invention,

said at least first aminopeptidase and said at least second aminopeptidase may be activated at a temperature above 80° C., and optionally comprising, prior to said contacting step, a step of denaturing the polypeptides of said substrate.

The term “contacting step” means a step in which the content of polypeptides of the substrate and the TET aminopeptidases of the composition may interact with one another, with a view to modifying the content of polypeptides of the substrate.

By “a first aminopeptidase and said at least second aminopeptidase which may be activated at a temperature above 80° C.” is meant that the aminopeptidases may pass from a stage in which they do not cleave the amino acids of the peptides, polypeptides and/or proteins of the substrate, to a stage where they cleave these amino acids.

The substrate polypeptides may first undergo a denaturation step, for example denaturation with propanol, which has the effect of promoting their modification by the aminopeptidases of the composition. These polypeptides are those which naturally have a tertiary structure. Consequently, organic solvents such as propanol cause destruction of the non-covalent bonds, for example the internal hydrogen bonds of the polypeptides. However, such bonds stabilize the tertiary structure of the polypeptides. Such destruction, therefore, causes destabilization, unfolding, or even denaturation of the polypeptides.

The invention also relates to a food compound capable of being obtained by the method of the invention.

The invention also relates to a food compound comprising at least one of the following proteins in modified form: gliadin, ß-lactoglobulin, α-lactalbumin, immunoglobulins, serum albumin and lactoferrin, said food compound further comprising a composition according to the invention.

The invention will be better understood in the light of the appended figures, which are provided by way of examples and are in no way limiting.

### BRIEF DESCRIPTION OF THE FIGURES

All the figures which follow represent chromatograms illustrating absorbance (expressed in mAu) as a function of an elution time (expressed in seconds).

FIGS. 1A, 1B and 1C are overlays of chromatograms resulting from the analysis in reverse phase chromatography (RP-HPLC) of the hydrolysis of synthetic peptide 2 incubated for 15 min with PhTET3 (FIG. 1A and curves A) and PhTET2 (FIG. 1B and curves C). The control sample, i.e. the peptide incubated without peptidase, is represented on curve B. FIG. 1C represents the overlay of the 3 chromatograms;

FIGS. 2A and 2B are overlays of the chromatograms resulting from the analysis in reverse phase chromatography (RP-HPLC) of the hydrolysis of synthetic peptide 4 incubated for 15 min with PhTET4 (FIG. 2A) and synthetic peptide 1 incubated for 15 min with PhTET1 (FIG. 2B);

FIGS. 3A, 3B, 3C and 3D are overlays of chromatograms resulting from analysis in reverse phase chromatography (RP-HPLC) of the hydrolysis of synthetic peptide 5 incubated for 15 min with PhTET3 (FIG. 3A and curves A), with MjTET (FIG. 3B and curves B) or with a mixture of 10% PhTET3/90% of MjTET (FIG. 3C and curves C). The control sample, i.e. the peptide incubated without peptidase, is represented in curves D. FIG. 3D represents the overlay of the 4 chromatograms;

FIGS. 4A, 4B, 4C and 4D are overlays of chromatograms resulting from analysis in reverse phase chromatography (RP-HPLC) of the hydrolysis of synthetic peptide 5 incubated for 15 min with PhTET3 (FIG. 4A and curves A), with MjTET (FIG. 4B and curves B) or with a mixture of 5% PhTET3/95% of MjTET (FIG. 4C and curves C). The control sample, i.e. the peptide incubated without peptidase, is represented in curves D. FIG. 4D represents the overlay of the 4 chromatograms;

FIGS. 5A, 5B and 5C are overlays of chromatograms resulting from analysis in reverse phase chromatography (RP-HPLC) of the hydrolysis of synthetic peptide 5 incubated for 15 min at different temperatures, at 40° C. (curves A) or 60° C. (curves B) with PhTET3 (FIG. 5A) with MjTET (FIG. 5B), or with a mixture of 5% PhTET3/95% of MjTET (FIG. 5C). The control sample, i.e. the peptide incubated without peptidase, is not visible in FIGS. 5A, 5B and 5C;

FIGS. 6A, 6B and 6C are overlays of chromatograms resulting from the analysis in reverse phase chromatography (RP-HPLC) of the kinetics of hydrolysis of synthetic peptide 7 incubated for 5, 15 or 30 min with PhTET4 (FIG. 6A), with MjTET (FIG. 6B) or with a mixture of 50% of PhTET4/50% of MjTET (FIG. 6C). Curves A: 5 min; curves B: 15 min; curves C: 30 min. The control sample, i.e. the peptide incubated without peptidase, is represented in curves D;

FIG. 7 shows an overlay of chromatograms resulting from the analysis in reverse phase chromatography (RP-HPLC) of the kinetics of the hydrolysis of synthetic peptide 7 incubated for 30 min with MjTET (curve A) or with a mixture of 50% of PhTET4/50% of MjTET (curve B). The control sample, i.e. the peptide incubated without peptidase, is represented on curve C;

FIG. 8 shows a chromatogram resulting from the analysis of the hydrolyzate of whey proteins in reverse phase chromatography (RP-HPLC);

FIG. 9 shows a overlay of the chromatograms resulting from the analysis of the hydrolyzate of whey proteins at pH=6.2 (solid line) and that at pH=9.5 (dotted line) in reverse phase chromatography (RP-HPLC). There are few differences between the hydrolyzate;

FIG. 10 shows an overlay of the chromatograms resulting from the analysis of the hydrolyzate of whey proteins at pH=6.2 alone (curve D), and after different incubations with different TETs. (Only part of the chromatogram is shown.). FIG. 10A shows the incubation with PhTET2 alone (curve A). FIG. 10B shows the incubation with PhTET3 alone (curve B). FIG. 10C represents the incubation with PhTET2 and PhTET3 in equimolar quantity (curve C);

FIG. 11 shows an overlay of the chromatograms resulting from the analysis of the hydrolyzate of whey proteins after incubation with PhTET2 and PhTET3 in equimolar amount (curves A) compared to different compositions of TET aminopeptidases. (Only part of the chromatogram is shown.). FIG. 11A represents the incubation with PhTET2 alone (curve B) and a mixture of 90% of PhTET2 and 10% of PhTET3 (curve 1). FIG. 11B represents the incubation with PhTET3 alone (curve C) and a composition of 10% of PhTET2 and 90% of PhTET3 (curve C1). FIG. 11C represents the incubation with a composition of 90% of PhTET2 and 10% of PhTET3 (curve 1) and with a composition of 10% of PhTET2 and 90% of PhTET3 (curve C1);

FIG. 12 shows an overlay of the chromatograms of FIGS. 11A, 11B and 11C, and the chromatogram resulting from the analysis of the hydrolyzate of whey proteins at pH=6.2 alone (curve D). Only part of the chromatogram is shown;

FIG. 13 shows an overlay of the chromatograms resulting from the analysis of the hydrolyzate of whey proteins at pH=9.5 alone (curve D), and after incubation with different TET aminopeptidases. (Only part of the chromatogram is shown.).

FIG. 13A shows the incubation with PhTET4 alone (curve A). FIG. 13B shows the incubation with MjTET alone (curve B). FIG. 13C represents the incubation with the composition of PhTET4 and MjTET in equimolar quantity (curve C);

FIG. 14 shows an overlay of the chromatograms obtained in reverse phase HPLC (column ZORBAX SB-300 C18). Curve A: sample of whey incubated in the presence of thermolysin. Curve B: sample of whey incubated in the presence of thermolysin and TET aminopeptidases. Curve C: sample of whey incubated alone. The column used here makes it possible to analyze small peptides, the “whole” proteins of the small are therefore not visible.

FIG. 15 shows an overlay of the chromatograms resulting from the analysis in reverse phase chromatography (RP-HPLC) of the hydrolysis of synthetic peptide 7 incorporated into a casein hydrolyzate incubated with PhTET3 (FIG. 15A), with MjTET (FIG. 15B), with PhTET4 (FIG. 15C) or with a composition of 33% of PhTET3/33% of PhTET4/33% of MjTET (FIG. 15D). Curve A: control (peptide 7 incorporated into the casein hydrolyzate without enzyme); curve B: PhTET3; curve C: MjTET; curve D: PhTET4; curve E: mixture of 33% of PhTET3/33% of PhTET4/33% of MjTET.

FIG. 16 represents an overlay of the chromatograms resulting from the analysis of gluten samples: non-incubated control (curve A), incubated without enzymes (curve B), incubated with the enzymes PhTET1, PhTET2 and PhTET3 in equimolar quantity (line VS). After 2 hours of incubation, there is a decrease in several absorbance peaks reflecting the significant degradation of several gluten proteins;

FIG. 17 represents an overlay of the chromatograms resulting from the analysis in reverse phase chromatography (RP-HPLC) of the hydrolysis of gluten samples. Curve A: sample of total gluten incubated in the presence of thermolysin. Curve B: sample of total gluten incubated in the presence of thermolysin and the TET aminopeptidases PhTET1, PhTET2 and PhTET3.

FIG. 18 shows an overlay of the chromatograms resulting from RP-HPLC analyzes of the control samples of the new whey protein hydrolyzate used for each mix.

FIG. 19 shows an overlay of the chromatograms resulting from the RP-HPLC analysis of the samples after hydrolysis of the new whey protein hydrolyzate by different mixtures of TET (mix 1 to 5).

FIG. 20 shows an overlay of the chromatograms resulting from the analysis in RP-HPLC of the hydrolysis of the peptides of the whey protein hydrolyzate by the mixture #1 (mix 1) of TET (70% PhTET2, 15% PhTET3, 15% PhTET4).

FIG. 21 shows an overlay of the chromatograms resulting from the analysis in RP-HPLC of the hydrolysis of the peptides of the whey protein hydrolyzate by the mixture #2 (mix 2) of TET (70% PhTET2, 15% PhTET4, 15% MjTET).

FIG. 22 shows an overlay of the chromatograms resulting from the analysis in RP-HPLC of the hydrolysis of the peptides of the whey protein hydrolyzate by the mixture #3 (mix 3) of TET (90% MjTET, 10% PhTET4).

FIG. 23 shows an overlay of the chromatograms resulting from the analysis in RP-HPLC of the hydrolysis of the peptides of the whey protein hydrolyzate by the mixture #4 (mix 4) of TET (70% MjTET, 15% PhTET1, 15% PhTET3).

FIG. 24 shows an overlay of the chromatograms resulting from the analysis in RP-HPLC of the hydrolysis of the peptides of the whey protein hydrolyzate by the mixture #5 (mix 5) of TET (70% MjTET, 15% PhTET3, 15% PhTET4).

FIG. 25 shows an overlay of the chromatograms resulting from the analysis in RP-HPLC of the hydrolysis of the peptides of the whey protein hydrolyzate by the mixture #6 (TET4-1 or mix 6) of TET (100% PhTET4).

FIG. 26 shows an overlay of the chromatograms resulting from the analysis in RP-HPLC of the hydrolysis of the peptides of the whey protein hydrolyzate by mixtures #1 and #2 of TET.

FIGS. 27 and 28 show an overlay of the chromatograms resulting from the analysis in RP-HPLC of the hydrolysis of the peptides of the whey protein hydrolyzate mixtures 1 and #2 of TET, compared to PhTET2 (“TET 2”).

### EXAMPLE

### Example 1

### Material and Methods

### TET Aminopeptidase Activity Test on Synthetic Peptides

In order to test the activity of TET aminopeptidases on synthetic peptides, different mixtures of TET aminopeptidases at a total concentration of 50 μg/ml are incubated with various peptides at a final concentration of 0.5 mM in a final volume of 100 μL. An internal standard is added to the experiments at the end of the reaction (not visible on the chromatograms shown), a sample of tryptophan at 50 μM. This internal standard is used to increase the accuracy of the calculations of the quantity of product in the medium. In other words, the quantity of standard injected into the column is precisely known, and it is thus possible to normalize the response signal obtained. The standard is a compound which does not react in the experiment and whose response to the signal is very close to the products measured. In this case, the internal standard chosen is tryptophan. The internal standard is added to the concentration indicated in the sample before its analysis in RP-HPLC. The activity tests are carried out at pH=7.5 in a 50 mM PIPES buffer, 150 mM KCl, except those in which PhTET4 is present which are carried out at pH=9.5 in a 50 mM CHES buffer, 150 mM KCl. The reaction medium is then incubated for different times at the desired temperatures (40° C. or 60° C.) with shaking (500 rpm). The tubes are then placed in ice to stop the hydrolysis reaction. Then, 80 μl of the reaction medium are added to 320 μl of a solution comprising 2% acetonitrile (ACN) and 0.1% trifluoroacetic acid (TFA). The samples are then centrifuged at 10,000 g for 10 min before being transferred to vials before their injection on an RP-HPLC column for analysis. In addition to their RP-HPLC analysis, the reaction media of these activity tests were also analyzed by mass spectrometry in order to precisely identify the size of the hydrolysis products observed. This made it possible to identify the different peaks observed on the chromatograms and to optimally follow the hydrolysis processes.

Preparation of a Protein Hydrolyzate from Cow's Whey

Whey represents the liquid fraction obtained after the coagulation of milk, and is a by-product obtained, in particular, in the cheese industry. It contains around 10% protein which is divided into 5 main families: β-lactoglobulin (50%), α-lactalbumin (20%), immunoglobulins (10%), bovine serum albumin (10%), and lactoferrin (2.8%). In the present case, these various proteins are hydrolyzed and the resulting peptides are used as a model substrate. A cow's whey solution is incubated in the presence of thermolysin (Sigma®) at a final concentration of 100 μg/ml for 2 h at 60° C. with shaking (500 rpm). After hydrolysis, the solution is incubated for 15 min at 95° C. in order to inactivate thermolysin. The whey protein hydrolyzate is then aliquoted and stored at −20° C. until use.

### TET Aminopeptidase Activity Test on a Whey Protein Hydrolyzate

In order to test the hydrolysis activity of the TET aminopeptidases on the peptides present in the whey protein hydrolyzate, various mixtures of TET proteins at a total concentration of 50 μg/ml, are incubated with the hydrolyzate in a final volume of 100 μl. No cofactor is added to the reaction. The activity tests of PhTET2 and PhTET3 were carried out on a whey protein hydrolyzate at its native pH of 6.2. The test series conducted with PhTET4 and MjTET was carried out at pH=9.5. The reaction medium is then incubated for 2 h at 60° C. with shaking (500 rpm). The tubes are then placed in ice to stop the hydrolysis reaction. Then, 80 μL of the reaction medium are added to 320 μl of a solution composed of 2% acetonitrile (ACN) and 0.1% trifluoroacetic acid (TFA). The samples are then centrifuged at 10,000 g for 10 min before being transferred to vials before their injection on an RP-HPLC column for analysis.

### Reverse Phase HPLC Analysis (RP-HPLC)

100 μl of each sample is injected either on a μRPC C2/C18 column (4.6 mm×100 mm) (GE Healthcare®) for studies on peptides, or on a ZORBAX SB-300 C8 column (4.6 mm×150 mm) (Agilent®) connected to a Perkin Elmer® HPLC system for studies on whey. Phase A consists of 0.1% TFA and 2% ACN in water, phase B contains 0.1% TFA and 80% ACN in water. The adsorbed proteins are then eluted at 1 ml/min with a linear gradient 0-50% of phase B, and are detected by measuring their absorbances at 280 nm for the studies on peptides or 214 nm for the other studies. Protein peaks are identified and analyzed using TotalChrom software version 6.3.1 (Perkin Elmer®).

### TET Aminopeptidase Activity Test Against Gluten Proteins

A 5% suspension of gluten proteins (Sigma®) is prepared using a metal stirrer in a solution allowing their solubilization (150 mM NaCl, 20 mM Tris-HCl, 50% propanol, pH=7.5). 95 μL of this solution containing the substrate are transferred into 0.5 ml Eppendorf tubes. The tubes are placed in an orbital shaker equipped with a thermostat, they are then incubated at 60° C. with shaking (500 rpm) for 10 min. A solution containing the mixture of the 3 enzymes PhTET1, PhTET2 and PhTET3 in equimolar quantity for a final concentration of 250 μg/ml is prepared and incubated under the same conditions. The reaction is begun by adding 5 μl of the mixture of enzymes to the reaction medium. The reaction is stopped after 2 h of incubation by placing the samples at 4° C.

### Reverse Phase HPLC (RP-HPLC) Analysis

The procedure is identical to that indicated above. However, the samples are deposited on a Jupiter C18 column (4.6 mm×200 mm) (Phenomenex), and the adsorbed proteins are then eluted with a linear gradient 0-40% of phase B.

### Results

The TET enzymes used in these experiments are metallo-aminopeptidases of the M42 family (MEROPS). They all belong to the same family of enzymes and have a very strong structural identity in them. On the other hand, they are In fact different enzymes with different specificities.

The three-dimensional structures of the various TETs used are very similar even though they all have specificities for different substrates. Thus, PhTET1 is a glutamyl-aminopeptidase, PhTET2 is a leukyl-aminopeptidase with significant residual activity towards some of the uncharged hydrophobic and polar residues, PhTET3 is a lysyl-aminopeptidase, PhTET4 is a strict glycyl-aminopeptidase, MjTET is a leucid with significant activity towards hydrophobic and positively charged residues. MjTET is also the only one to have hydrolysis activity towards aromatic residues.

All of the results presented above were obtained using substrates of the monoacyl-pNA (4-nitroaniline) or monoacyl-AMC (7-amino-4-methylcoumarin) type. They represent a peptide of two residues, the first residue is the one on which the activity of the peptidase is to be measured, the second is a chromophore which emits a signal in the visible when released. Thus, we may measure the affinity of a peptidase for each of the known residues.

Thus, several peptides with specific sequences have been designed and synthesized (Table 1 below). On the one hand, 5 peptides of 15 residues called “enriched”, i.e. the N-terminal end of these peptides has been enriched in a particular type of residue:

TABLE 1
List of synthetic peptides used during the st

[symbol]

Peptide 1: enriched in negatively charged residues
Peptide 2: enriched in hydrophobic residues
Peptide 3: enriched in positively charged residues
Peptide 4: enriched in glycines
Peptide 5: enriched with aromatic residues

| | 1 | 2 | 3 | 4 | 5 | 6 | 7 | 8 | 9 | 10 | 11 | 12 | 13 | 14 | 15 | |
|---|---|---|---|---|---|---|---|---|---|---|---|---|---|---|---|---|
| | xxx | xxx | xxx | xxx | xxx | xxx | xxx | xxx | xxx | xxx | xxx | xxx | xxx | xxx | xxx | |
| # Peptide | Enriched sequence | | | | Compensated sequence | | | | | Common sequence | | | | | | |
| 1 | Glu | Glu | Asp | Glu | Lys | Arg | Arg | Lys | 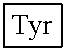 | Thr | Ser | 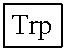 | Asn | Ser | Glu | SEQ ID NO: 8 |
| 2 | Leu | Met | Leu | Leu | Asn | Glu | Asn | Arg | 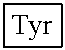 | Thr | Ser | 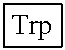 | Asn | Ser | Glu | SEQ ID NO: 9 |
| 3 | Lys | Arg | Arg | Lys | Glu | Glu | Asp | Glu | 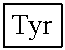 | Thr | Ser | 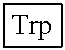 | Asn | Ser | Glu | SEQ ID NO: 10 |
| 4 | Gly | Gly | Gly | Gly | Asn | Glu | Asn | Arg | 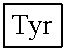 | Thr | Ser | 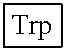 | Asn | Ser | Glu | SEQ ID NO: 11 |

| | | | | | | | | | | | | | | | |
|---|---|---|---|---|---|---|---|---|---|---|---|---|---|---|---|
| 5 | 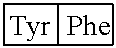 | Leu | Met | Asn | Glu | Asn | Arg | 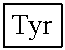 | Thr | Ser | 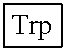 | Asn | Ser | Glu | SEQ ID NO: 12 |

Random Peptides

| | | | | | | | | | | | | | | | | |
|---|---|---|---|---|---|---|---|---|---|---|---|---|---|---|---|---|
| 6 | Glu | Tyr | Asn | Lys | Ala | Gly | Arg | Thr | Glu | Leu | Phe | Gln | Ile | Ser | Val | SEQ ID NO: 13 |
| 7 | Gly | Ile | Ser | Glu | Gln | Phe | Lys | Thr | Asn | Val | Leu | Arg | Glu | Tyr | Ala | SEQ ID NO: 14 |
| 8 | Phe | Lys | Tyr | Val | Gly | Gln | Asn | Glu | Ile | Ser | Arg | Ala | Thr | Leu | Glu | SEQ ID NO: 15 |
| 9 | Lys | Leu | Val | Arg | Glu | Ile | Tyr | Glu | Gln | Phe | Asn | Gly | Thr | Ala | Ser | SEQ ID NO: 16 |
| 10 | Leu | Asn | Glu | Gly | Phe | Thr | Glu | Lys | Gln | Ser | Val | Ala | Arg | Ile | Tyr | SEQ ID NO: 17 |

[symbol]

indicates data missing or illegible when filed

Each of these peptides carries an enriched N-ter end. Then follows 4 residues which compensate for the effect of the enriched zone (in particular for the solubility of the peptide). The C-terminal end is conserved and carries the same series of 7 residues chosen for their absorbance at 280 nm and their solubility: YTSWNSE (SEQ ID NO: 7).

On the other hand, 5 so-called “random” peptides, i.e. they contain 15 identical residues but distributed according to different sequences: Peptide 6; Peptide 7; Peptide 8; Peptide 9; Peptide 10.

During the various experiments carried out with the TET aminopeptidases, using the above peptides as substrates, several unexpected activities were observed by the inventors.

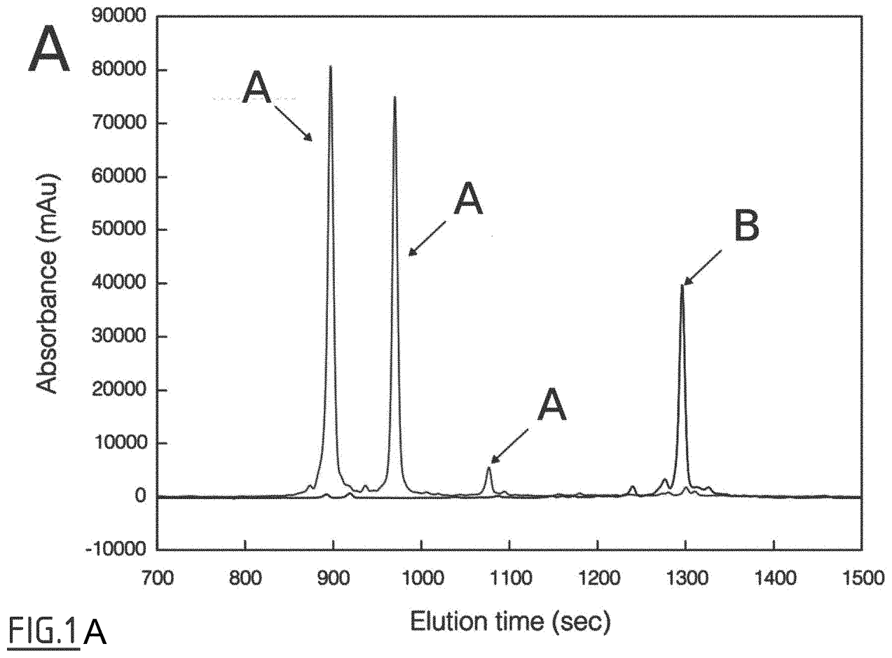

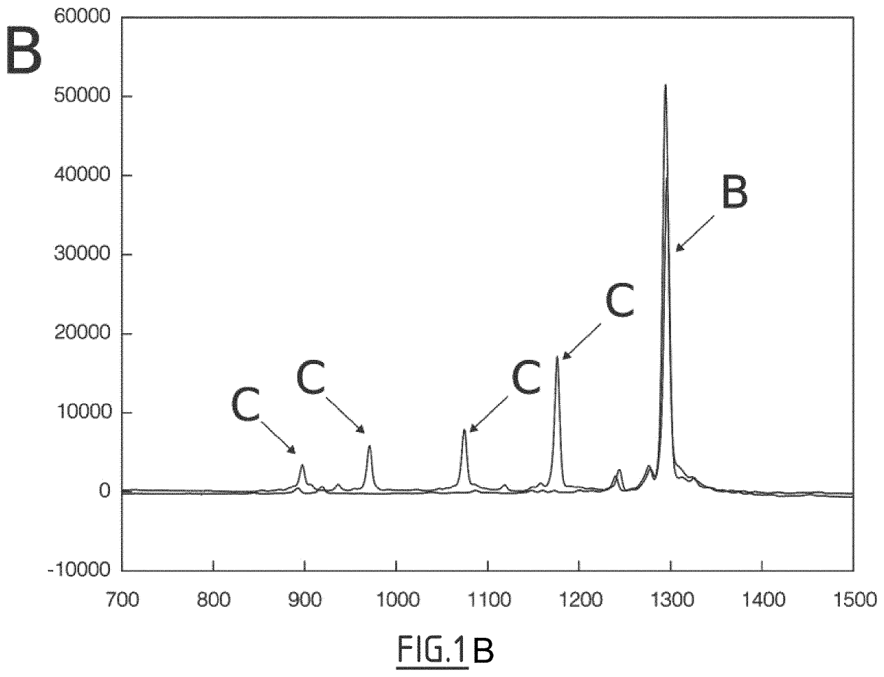

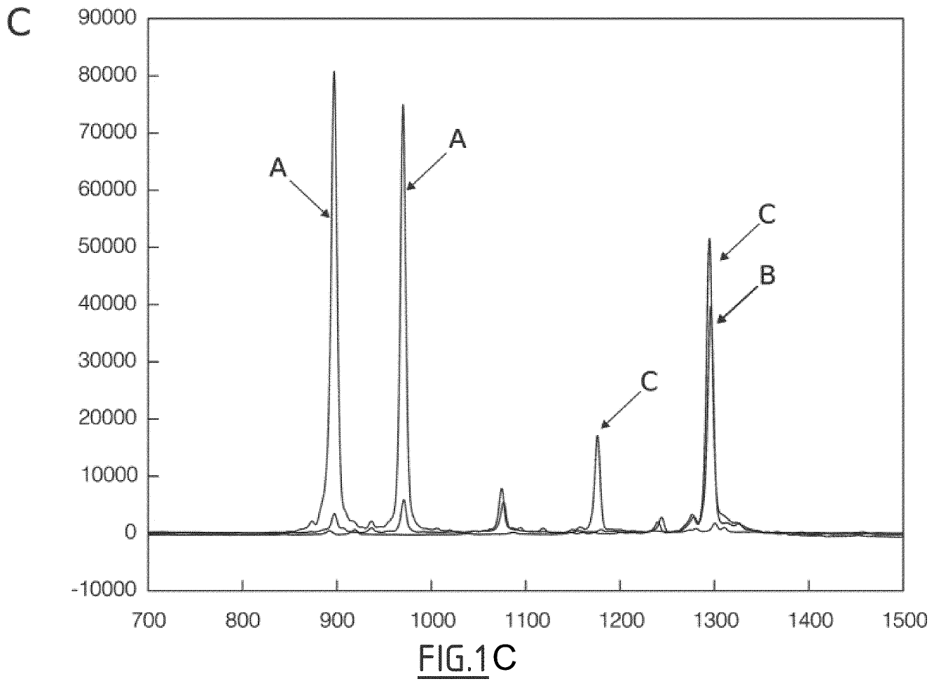

Aminopeptidase PhTET3 exhibits optimal activity against positively charged residues; it is classified as a lysyl-aminopeptidase with residual activity against leucine, methionine, glutamine and aspartate. However, during tests carried out on synthetic peptides, significant activity of PhTET3 was observed on the peptide enriched in hydrophobic residues, peptide 2 (FIG. 1A).

The same test was carried out with the aminopeptidase PhTET2 (FIG. 1B), which, according to the prior art, is the most effective of the aminopeptidases PhTET (i.e. aminopeptidases originating from thermococcals) against hydrophobic residues.

It is remarkable to note that PhTET3 exhibits greater activity on the peptide enriched in hydrophobic residues than PhTET2 (FIG. 1C). In fact, during the 15 minutes of the activity test, the first residues of the peptide were hydrolyzed more quickly in the presence of PhTET3 than in the presence of PhTET2.

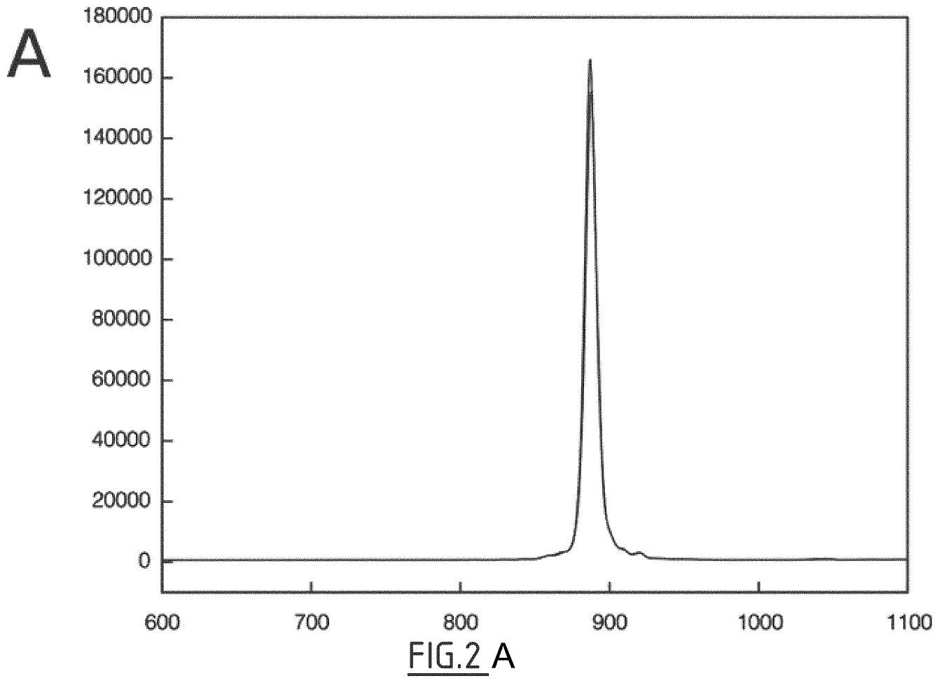

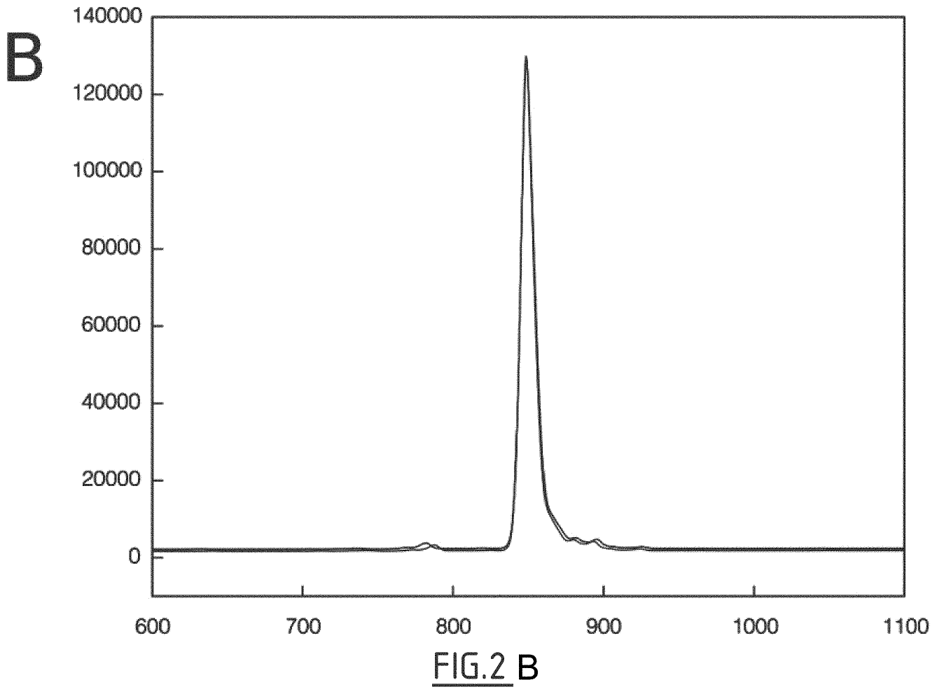

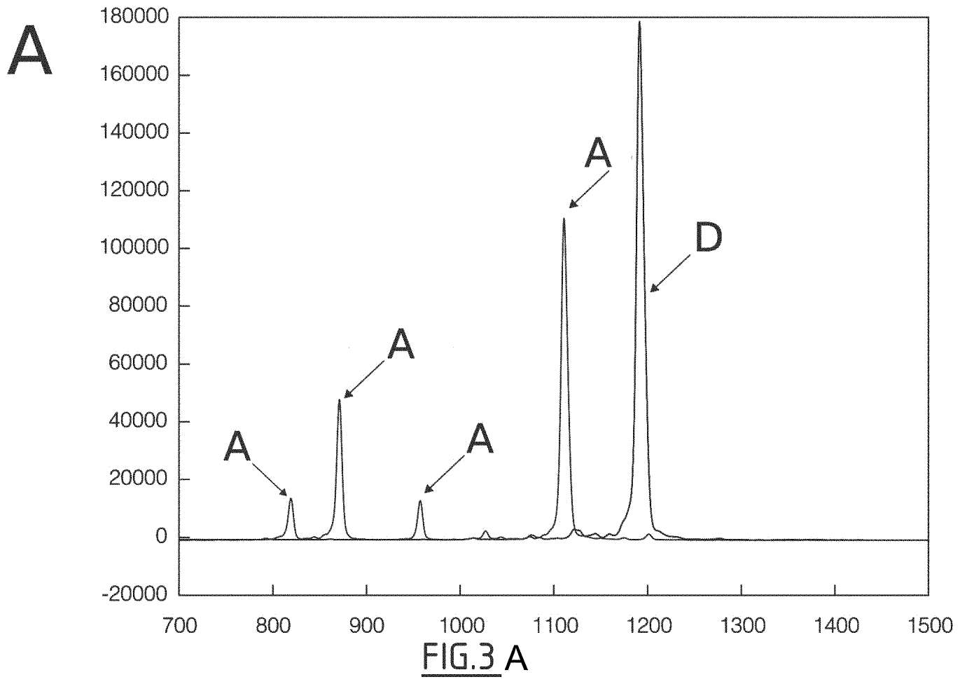

This type of “unexpected” activity is also observed in the case of peptides 1 and 4 respectively enriched in glutamic acid and glycine residues. When these are incubated in the presence of peptidases which, in theory, exhibit the maximum hydrolysis activity against the residues whose ends are enriched, no hydrolysis activity is observed (FIGS. 2A and 2B).

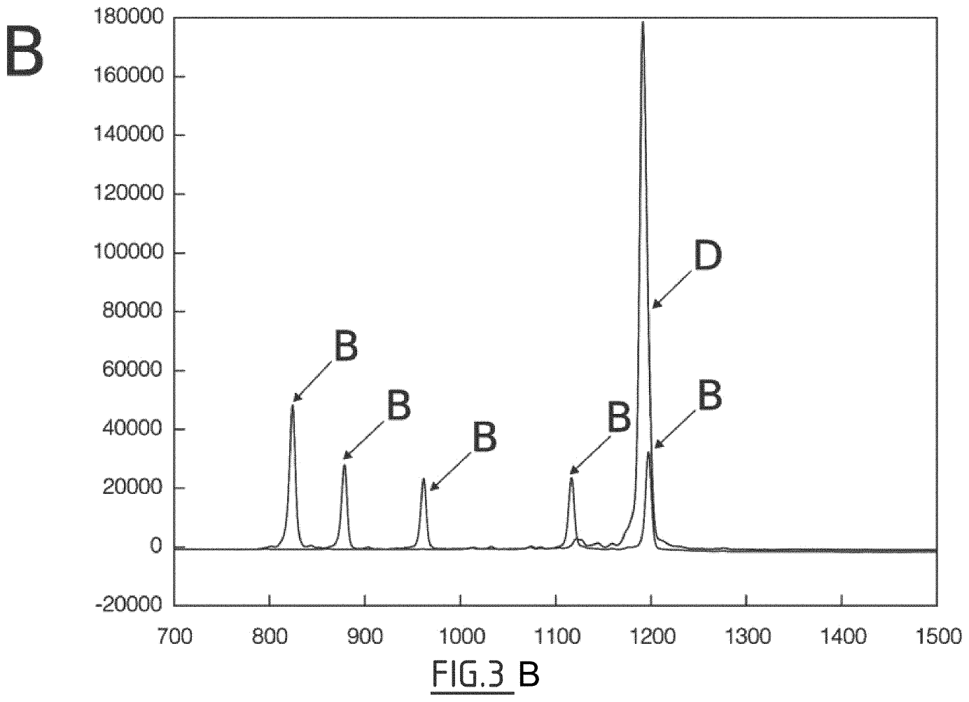

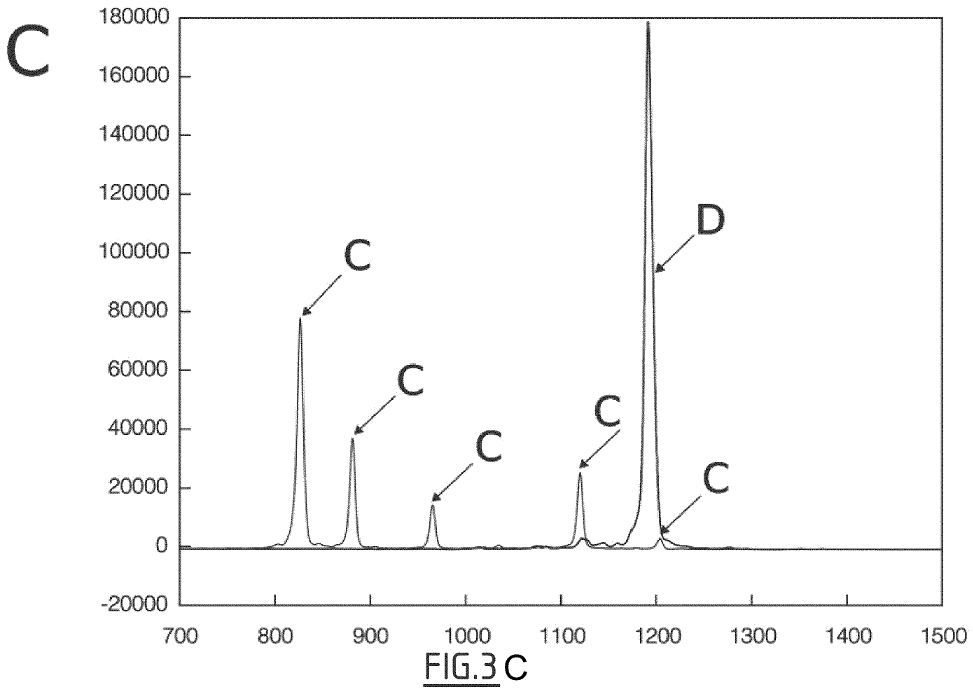

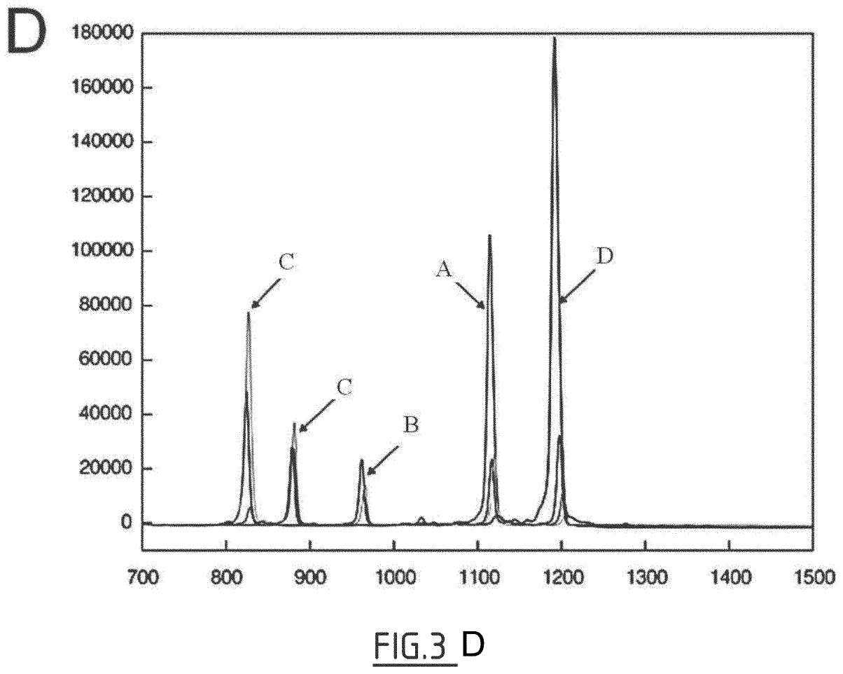

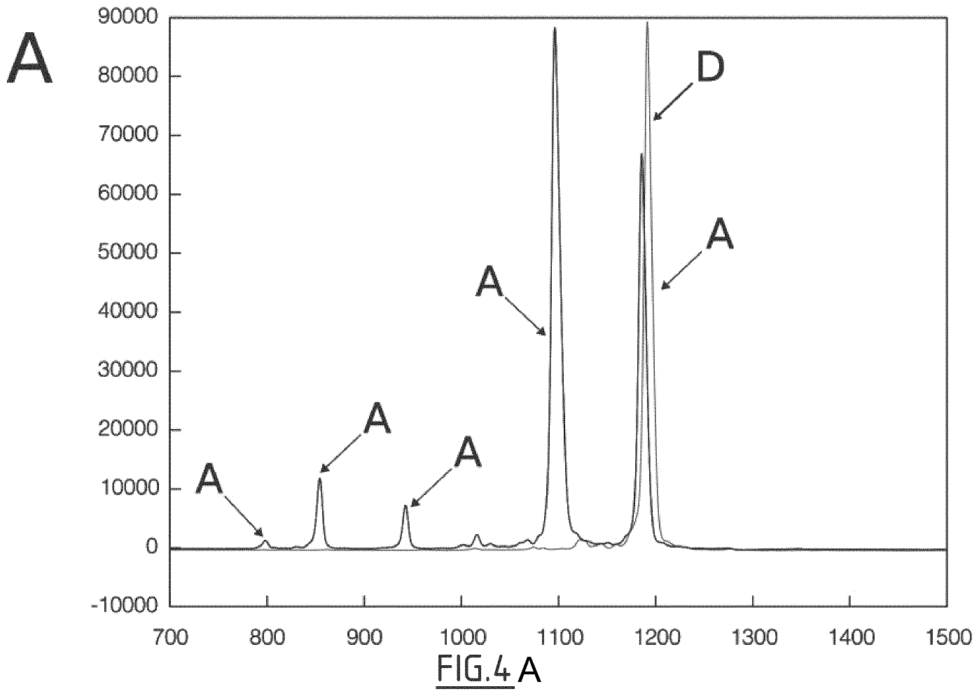

Another unexpected result was observed when the activity of PhTET3 was measured on a peptide whose N-terminal end is enriched in aromatic residues (FIG. 3A). It is quite surprising, given the data available in the literature, to observe the hydrolysis of aromatic residues by PhTET3. In fact, the analysis of the different peaks of proteins eluted by mass spectrometry shows a significant accumulation of peptides whose tyrosine at the N-terminal end has been hydrolyzed.

Until now, the characterization of the enzyme specificities of TET aminopeptidases has been obtained by analyzing their activity against dipeptides, these are the data available in the literature. Tests for the activity of PhTET3 on synthetic peptides 2 and 5 show that, in reality, the activity of TET aminopeptidases may be dependent on the nature of the substrate peptide. In fact, even though PhTET3 is not capable of hydrolyzing a tyrosine residue carried by a Tyr-pNA dipeptide, the latter shows great hydrolysis efficiency against this residue in the context of peptide 5. To date, no unexpected activities of the same type have yet been shown; however, it is not excluded that tests on a larger scale, i.e. with a greater variety of substrate, might reveal other activities.

The results of FIGS. 1A, 1B, 1C, 2A and 2B show that, despite the fact that some of these enzymes have been described, their integration into enzymatic compositions is not trivial. In fact, in addition to the theoretical specificities of these aminopeptidases, the nature of the food peptide to be modified and the residues surrounding the site to be modified, in addition to the physico-chemical parameters of the reaction medium, must be taken into account when designing specific enzymatic compositions. These conclusions, as well as the advantages of using compositions according to the invention are highlighted in the experiments which follow.

### Accelerated Hydrolysis and Modulation

### MjTET 90%/TET3 10%

In order to demonstrate the interest of TET aminopeptidase compositions for improved hydrolysis of peptides, the activity of a composition of 10% PhTET3 and 90% MjTET was tested against peptide 5, rich in aromatic residues (FIGS. 3A-3D). Each aminopeptidase was incubated alone with the substrate peptide; the reaction medium analyzed by RP-HPLC shows that the two aminopeptidases are active on the peptide. In the reaction medium, 4 degradation products are identified, corresponding to the substrate peptide from which one (pep-1), two (pep-2), three (pep-3), or four (pep-4) residues has/have been hydrolyzed in addition to the intact substrate (pep-0).

In the case of PhTET3 (FIG. 3A), there is an accumulation of the peptide pep-1 while the intact substrate peptide is no longer present in the medium.

In the case of the peptide MjTET (FIG. 3B), part of the intact peptide is still present in the reaction medium, while the residues of the hydrolysis products are present in almost equivalent amounts. There is a slight accumulation of the final hydrolysis product, the peptide pep-4.

After incubation of the peptide in the presence of the composition of the two aminopeptidases, the substrate peptide is no longer present in the medium and there is a greater accumulation of the peptide pep-4, signifying that the hydrolysis of peptide 5 has been more effective in the presence of the composition comprising the TET aminopeptidases than when the aminopeptidases are used alone (FIG. 3C). The chromatograms resulting from the three tests are superimposed on FIG. 3D, and it may thus be clearly observed that the addition to the mixture of 10% of the aminopeptidase PhTET3 (alone) made it possible to significantly accelerate the hydrolysis of a peptide, even though this does not have an optimal activity on the peptide.

### MjTET 95%/TET3 5%

The same experiment was carried out by modifying the ratio of the TET3/MjTET mixture; this time the peptide was incubated in the presence of a composition of 95% of MjTET and 5% of PhTET3.

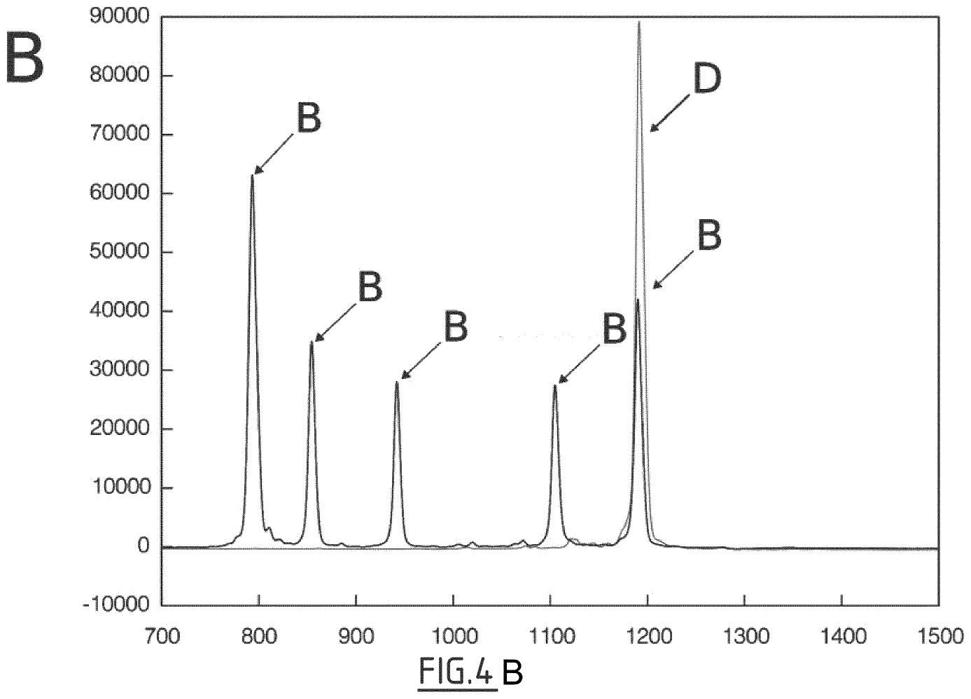

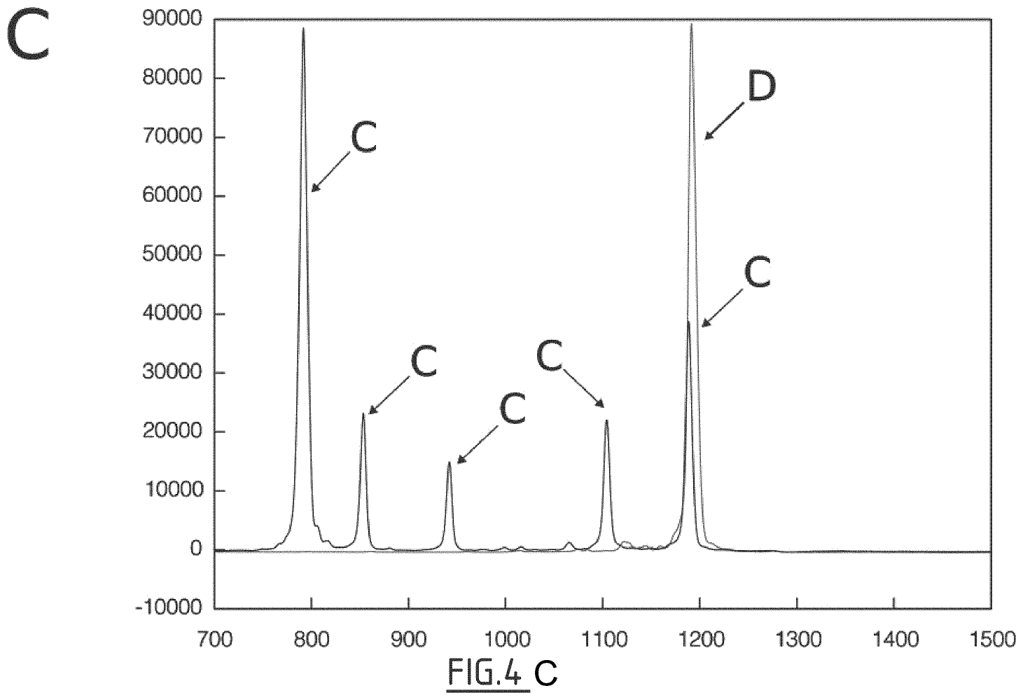

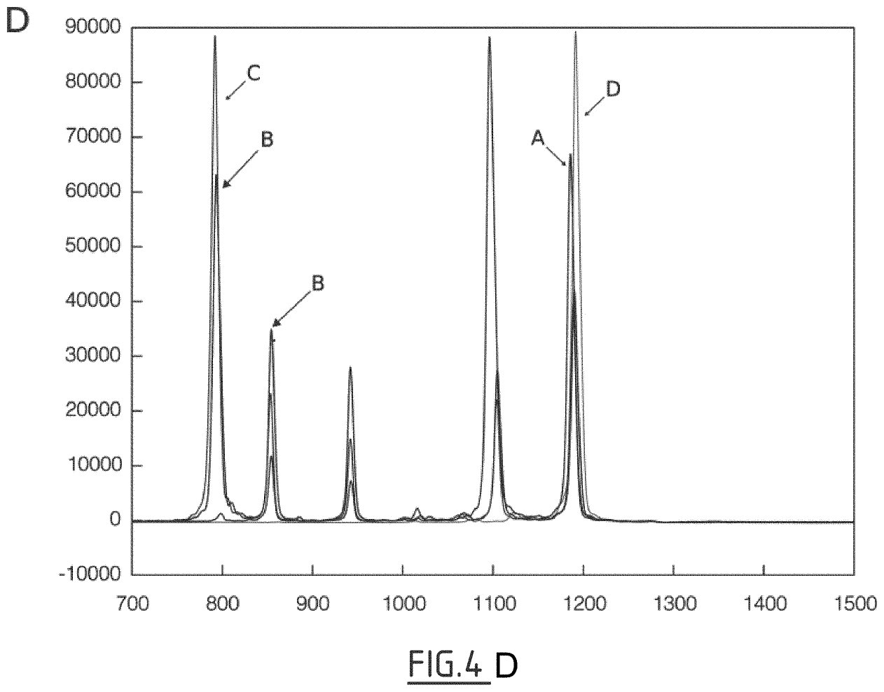

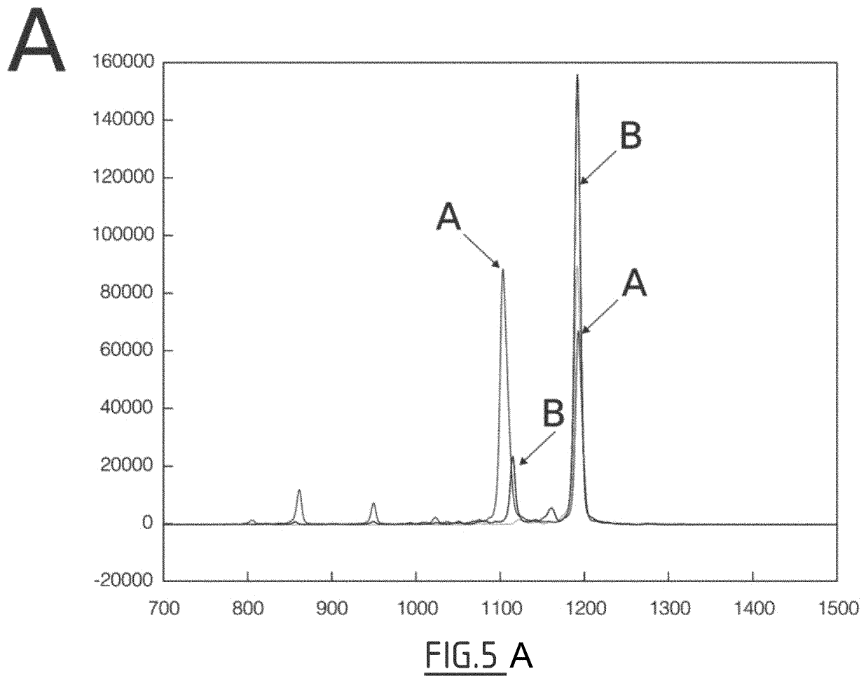

Again, in the case of PhTET3, there is a strong accumulation of the peptide pep-1 after the 15 minutes of incubation of the aminopeptidase with the substrate peptide (FIG. 4A). The intact peptide pep-0 is itself still present in the reaction medium in a substantial amount. The hydrolysis profile of peptide 5 by MjTET present at 95% (FIG. 4B) is close to that obtained at 90% (FIG. 3B).

These data, compared with those obtained with the composition at 95% of MjTET and 5% of PhTET3 (FIG. 4C), show once again an acceleration of the hydrolysis of the peptide by adding the aminopeptidase PhTET3 to the reaction mixture. (FIG. 4D). In cases where the enzymes are used alone, it is observed that the intermediate reaction products, pep-1, pep-2 and pep-3, are accumulated. In this last experiment, there is a decrease in the concentration of these intermediates, while there is an increase in that of the final product pep-4. Adding 5% of the peptide PhTET3, therefore, makes it possible to speed up the hydrolysis process of the peptide.

It is all the more interesting to note that in this specific case, the addition of an aminopeptidase which is, in theory, not specific for the residues which it is desired to hydrolyse makes it possible to increase the general efficiency of the composition of TET aminopeptidases.

Finally, these two examples of TET aminopeptidase compositions, with modification of the ratio, show the possibility offered by the compositions according to the invention in terms of hydrolysis modulation, in this case allowing modification of the peptides substrates rather than destroying them completely.

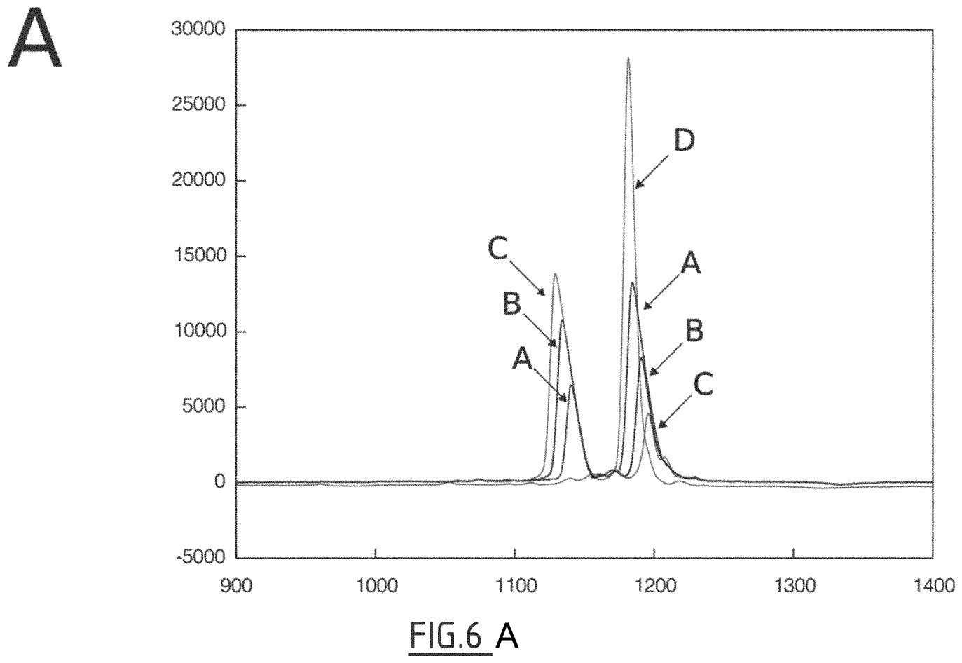

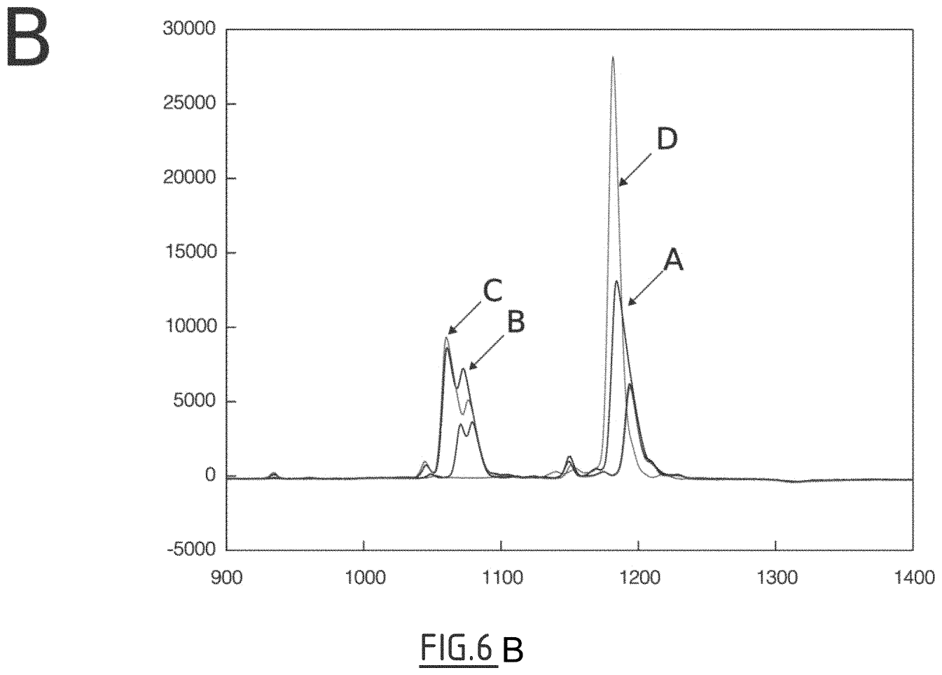

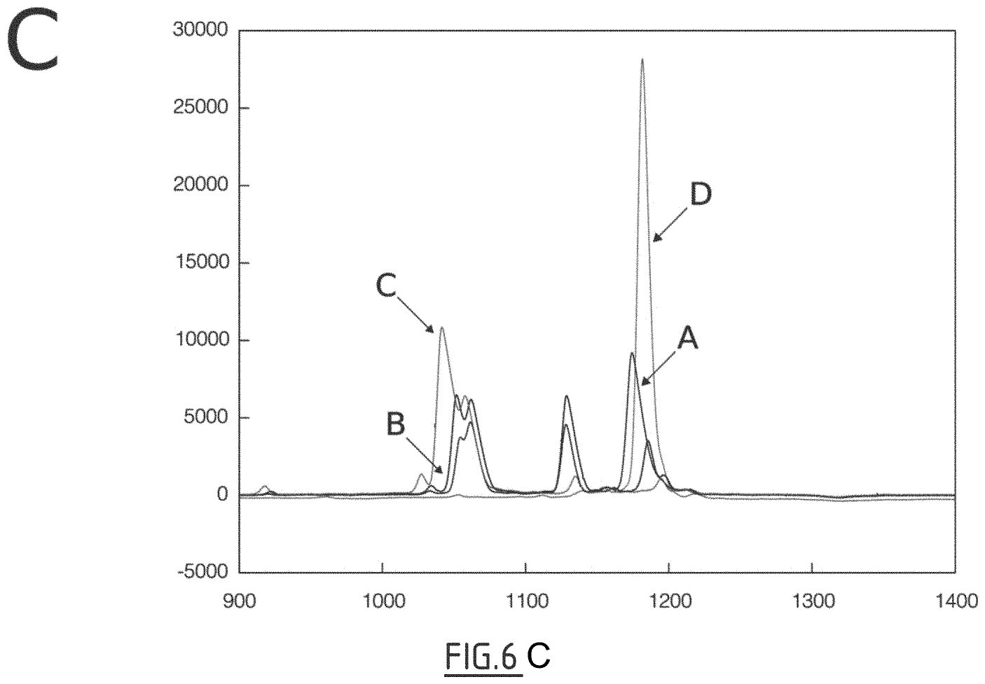

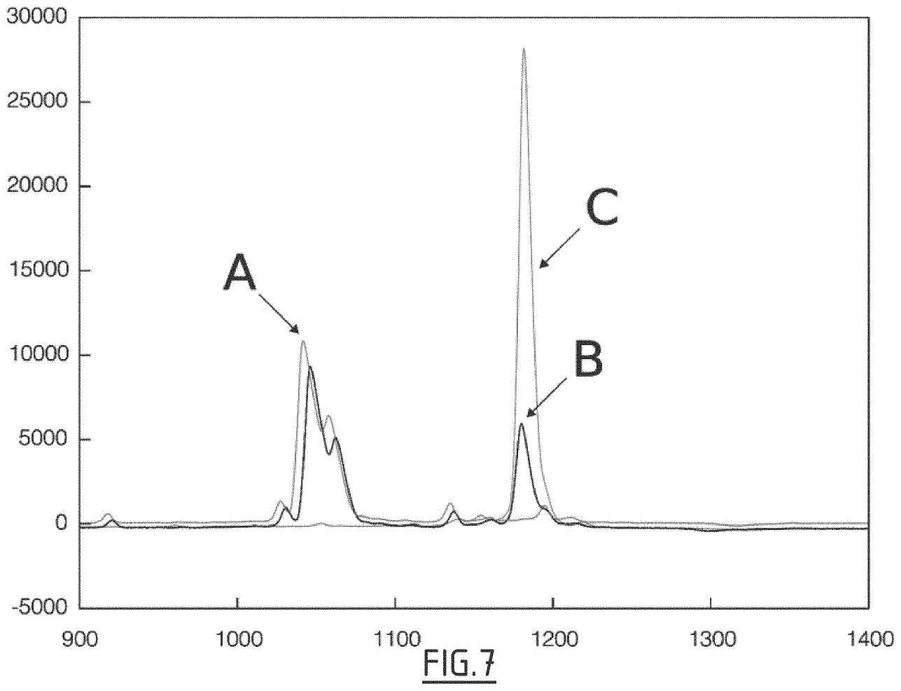

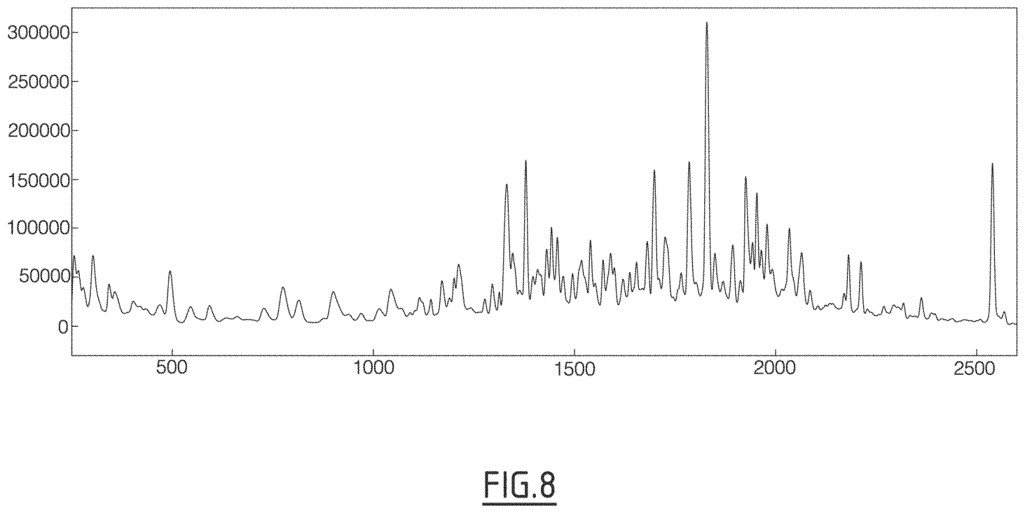

The experiments presented above were carried out at 60° C. They were also carried out at 40° C. and the data obtained were then compared to the previous data (FIG. 6).

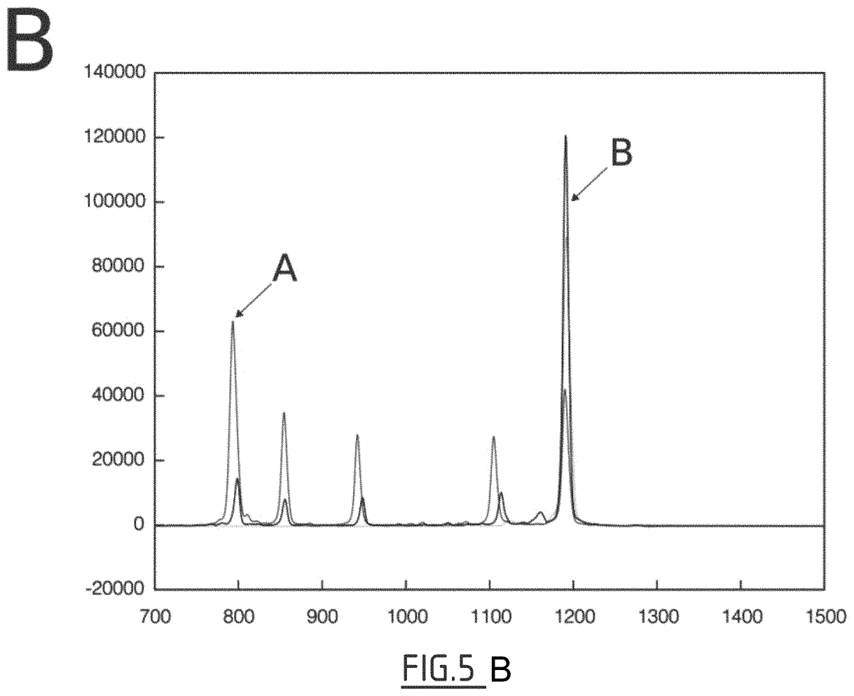

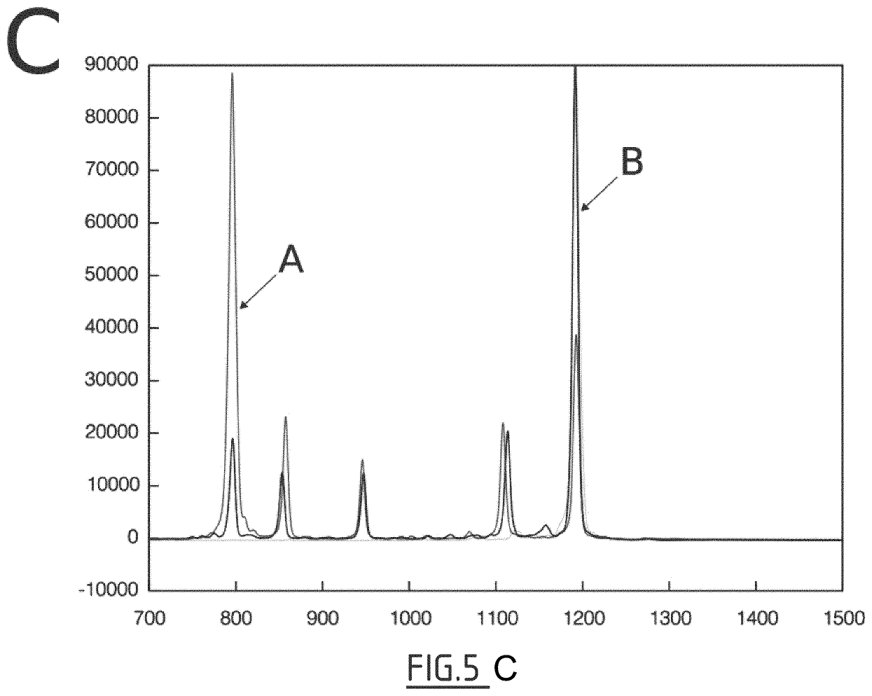

When the PhTET3 aminopeptidase is incubated alone with the substrate peptide at 40° C., a significant decrease in hydrolysis is observed. In fact, in this case, only the peptide pep-1 is visible and in very small quantity (FIG. 5A).

On the other hand, the same effect is observed in the case of MjTET, wherein all the intermediate peptides are visible as well as the final product pep-4, and there is only a significant slowdown in hydrolysis (FIG. 5B).

In the case of a 95% MjTET and 5% PhTET3 composition (FIG. 5C), the concentration of the final product pep-4 is greatly reduced. On the other hand, it is interesting to note that the various intermediates pep-1, pep-2 and pep-3 are, in turn, present in an amount almost equivalent to hydrolysis at 60° C. Thus, the hydrolysis on the first residue, namely tyrosine, is as effective at 40° C. as at 60° C. when the TET composition is used, whereas the latter is greatly reduced when the aminopeptidases are used alone. On the other hand, the rest of the hydrolysis process seems to be slowed down, leading us to the observed result where the substrate peptide is still present in large quantities, while the final product, on the contrary, has accumulated very little.

This example once again demonstrates the modulation which it is possible to integrate into the method for modifying the polypeptide content of a substrate according to the invention. In this part, modulations of quantities (different ratios of TET aminopeptidases) and temperatures were shown. It is also possible to integrate pH and time modulations, in order to obtain finer and more precise peptide modifications. These examples were carried out with two TET aminopeptidases. However, the addition of other TET aminopeptidases makes it possible to target peptides of interest more broadly, or more precisely.

### MjTET/PhTET4 Compositions on Random Peptide

The kinetics of hydrolysis of random peptide 7 by a composition of the aminopeptidases MjTET and PhTET4 was measured (FIG. 6). The hydrolysis of the peptide was analyzed by RP-HPLC after 5 min, 15 min and 30 min. Peptide 7 has the particularity of presenting a glycine at the N-terminal end.

The characterization of the aminopeptidase PhTET4 has shown that it is a glycine-aminopeptidase. This is why, when its activity was tested on peptide 7, only the hydrolysis of the first glycine residue was observed (FIG. 6A). We can thus see the accumulation of the peptide pep-1.

In the case of hydrolysis of the peptide by the aminopeptidase MjTET, we observe, in addition to the peptide pep-1, the peptides pep-2 and pep-3, and what corresponds to pep-4 (FIG. 6B). On the other hand, it is noted that between 15 and 30 min, the concentration of the substrate peptide pep-0 does not seem to have decreased. At the same time, we note that the concentration of pep-2 has decreased, while that of pep-3 has increased. The phenomenon observed here is due to the fact that the affinity of the aminopeptidase for the hydrolysis product is greater than that of the starting substrate.

When a composition, comprising the two aminopeptidase PhTET4 and MjTET in equimolar proportions is brought into contact with the substrate peptide and incubated, a significant improvement in the hydrolysis of the peptide is observed (FIG. 6C). The concentration of the intact peptide pep-0 gradually decreases between 5 and 30 min. That of the peptide pep-1, on the other hand, increases initially between 5 and 15 min before decreasing sharply between 15 and 30 min.

The chromatograms obtained after 30 min of incubation with MjTET alone or with the composition MjTET and PhTET4 are overlaid in FIG. 7. It is thus observed that the final peptides are present in relatively similar amounts, while the substrate peptide has been almost completely hydrolyzed. It is noted that, alone, neither of the two peptidases was able to completely hydrolyze the first residue of the peptide during the experiment. Tests with a third peptidase, PhTET3, were carried out.

In this example, the hydrolysis of the first residue of the substrate peptide is accelerated by the addition to the composition of a peptidase which, again, did not exhibit optimal activity. It should be noted that by modulating the TET aminopeptidase type, their ratio, the temperature or even the pH in a specific way, the modification method makes it possible to enrich a mixture of peptide with one of the observed hydrolysis intermediates (in this case the pep-2 peptide enriched at 15 min in FIG. 6B).

### Modulation of Peptide Hydrolysis in a Complex Mixture

### Whey Protein Hydrolyzate

Whey represents the liquid fraction obtained after coagulation of milk. It contains approximately 10% protein which is divided into 5 main families: ß-lactoglobulin (50%), α-lactalbumin (20%), immunoglobulins (10%), bovine serum albumin (10%) and lactoferrin (2.8%).

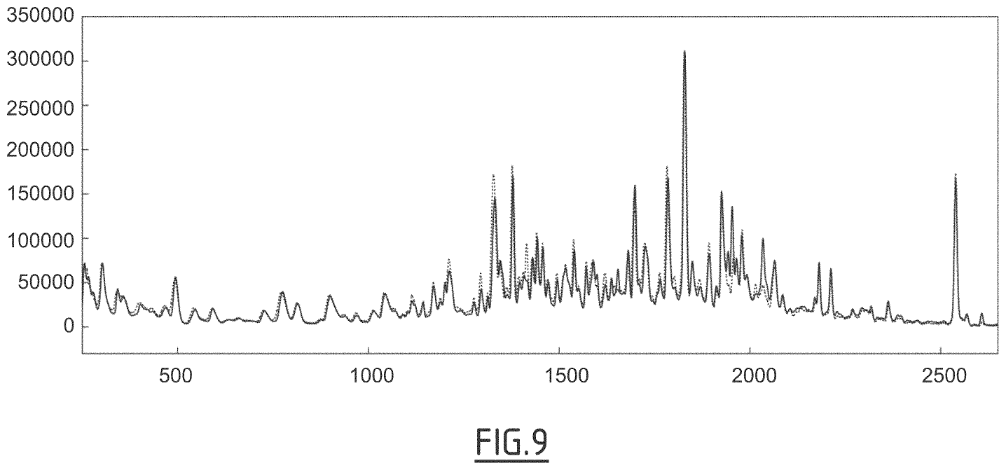

The substrate used in the following tests is a whey protein hydrolyzate, the preparation of which is explained above. In order to analyze the relative peptide composition of the hydrolyzate, it is analyzed by reverse phase chromatography on an HPLC system. The chromatogram resulting from the analysis of the control hydrolyzate is shown in FIG. 9.

Two hydrolyzates were prepared at different pH levels in order to be able to analyze the activity of the TET enzymes on these peptides under their optimal activity conditions. The two chromatograms are overlaid in FIG. 9.

It is thus observed that the general appearance of the chromatogram remains unchanged. There was, therefore, no drastic change in the peptide composition during the change in pH. However, there are fine changes in the elution profile. These ad hoc changes were taken into account in the analysis of the results.

### PhTET2/PhTET3 Compositions (50/50%, 90/10% and 10/90%)

Here, the possibility of modulating the hydrolysis of specific peptides within a complex mixture is demonstrated when the TET aminopeptidases are used in characteristic compositions like those of the invention.

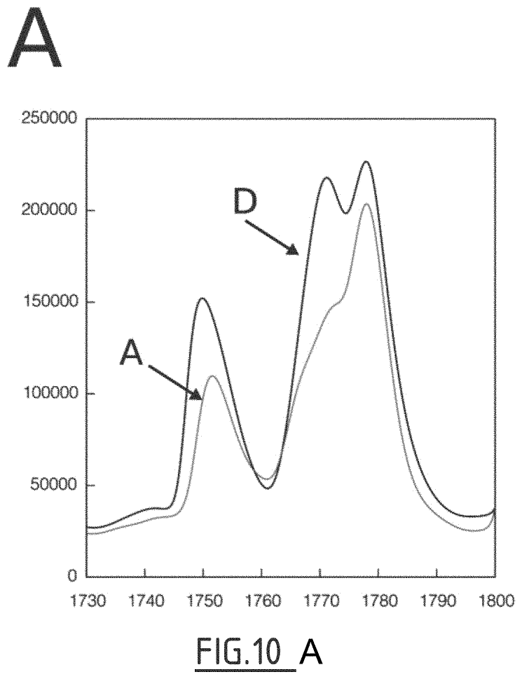

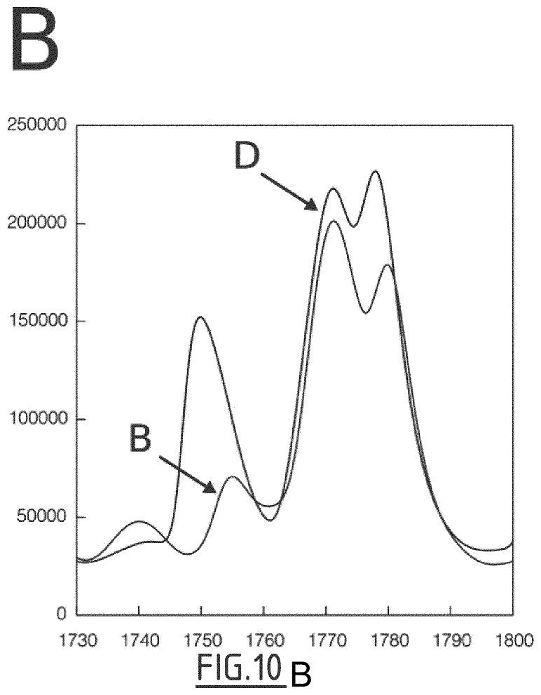

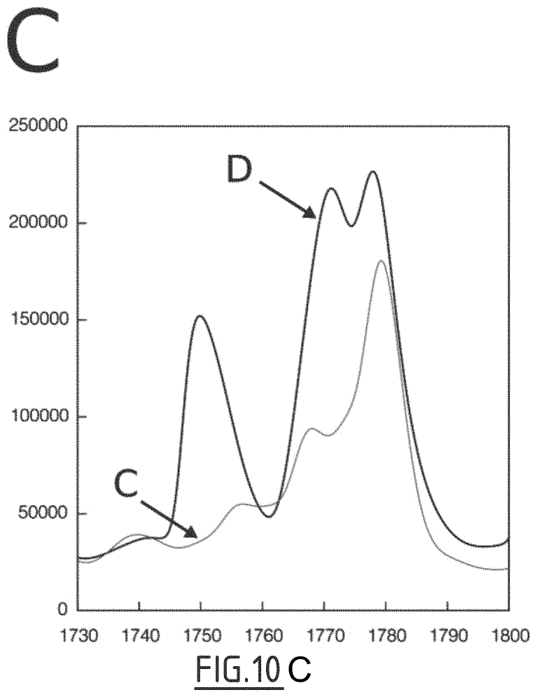

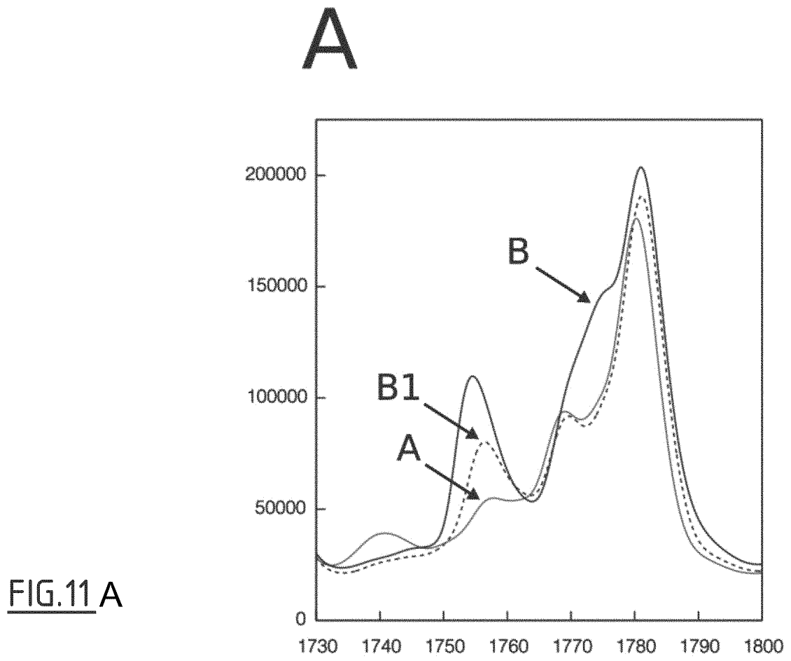

The activities of PhTET2 and PhTET3 are first measured when the aminopeptidases are used alone (FIG. 10). It is observed that not all the peptides are taken up by the TET aminopeptidases, this being due to the fact that the two TET aminopeptidases used have specificities of different and marked substrates. The mixture of peptides used for this experiment being a complex mixture, we also observe differences in the degree of hydrolysis of the different peptides supported by the aminopeptidases. Only part of the chromatogram is shown for clarity; the results presented may, however, be observed on the whole chromatogram.

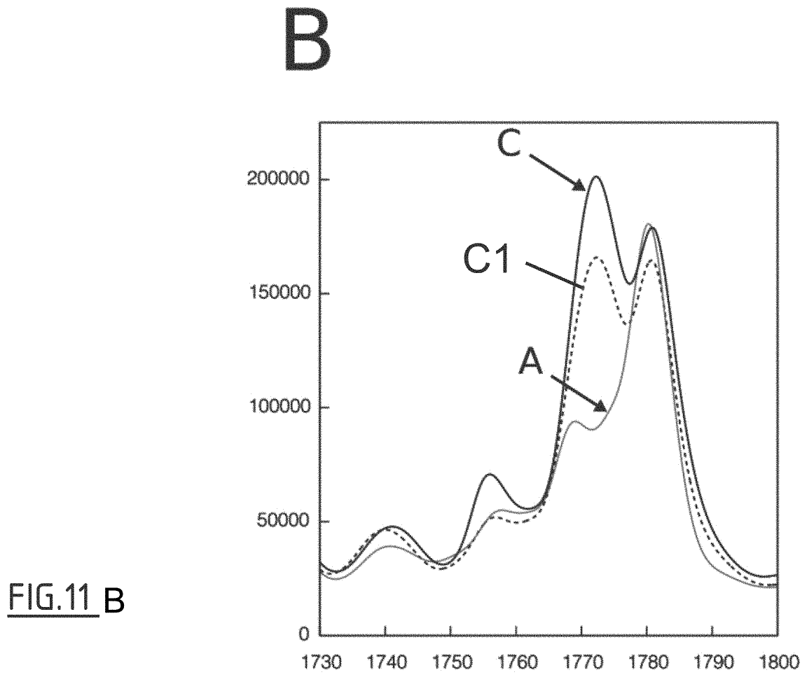

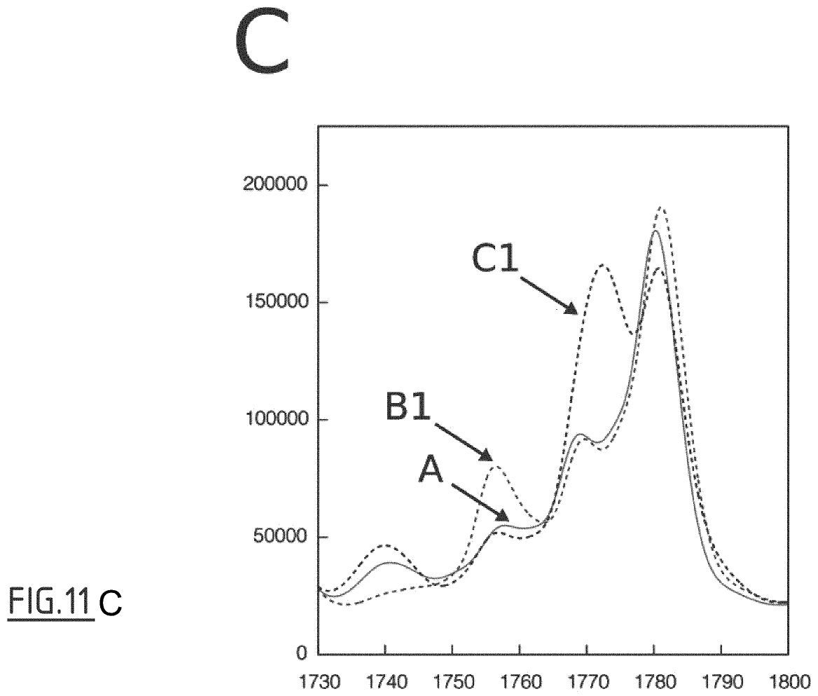

When the hydrolyzate is incubated with a composition of 2 aminopeptidases in equimolar amounts, an improvement in the hydrolysis activity is then observed on most peptides in solutions. Unexpectedly, the degree of hydrolysis changes by modifying the ratio of the different TETs in the composition (FIG. 11).

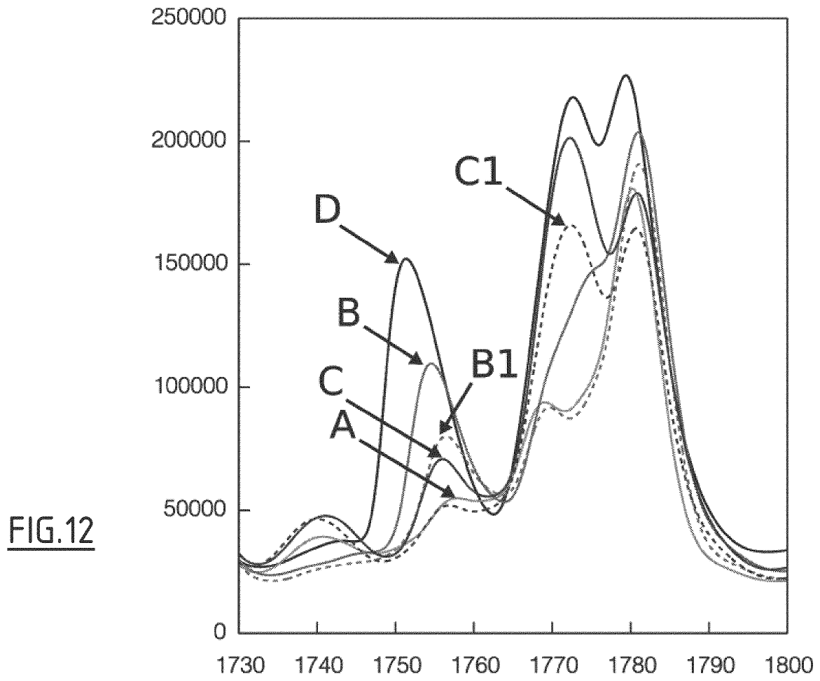

FIG. 12 shows the chromatograms obtained with 3 different ratios of compositions of PhTET2 and PhTET3. First, the “equimolar” mixture corresponding to 50% of each of the TETs, it is also visible in FIG. 10. The chromatograms shown in dotted lines correspond to different ratios of each TET aminopeptidase, 90% of the one and 10% of the other, and vice versa. It is thus noted that the hydrolysis profiles are modified, meaning that a variation in the proportions of each TET aminopeptidase in the mixture modifies the degree of hydrolysis of the peptides in solutions. An overlay of all the different chromatograms is presented in FIG. 12 and makes it possible to clearly visualize the possible modulation in the hydrolysis of the peptides.

This is a surprising result, insofar as it was impossible to predict. Furthermore, it is noted that the modulation of hydrolysis varies depending on the peptides; thus, it is possible by finely modifying the proportions of each TET aminopeptidase in the composition, to modulate the hydrolysis of the various peptides. No information in the prior art could suggest that it was possible to modulate the hydrolysis of peptides in a targeted manner by varying the concentration of the different TETs in a mixture.

### MjTET/PhTET4 Compositions (50/50%)

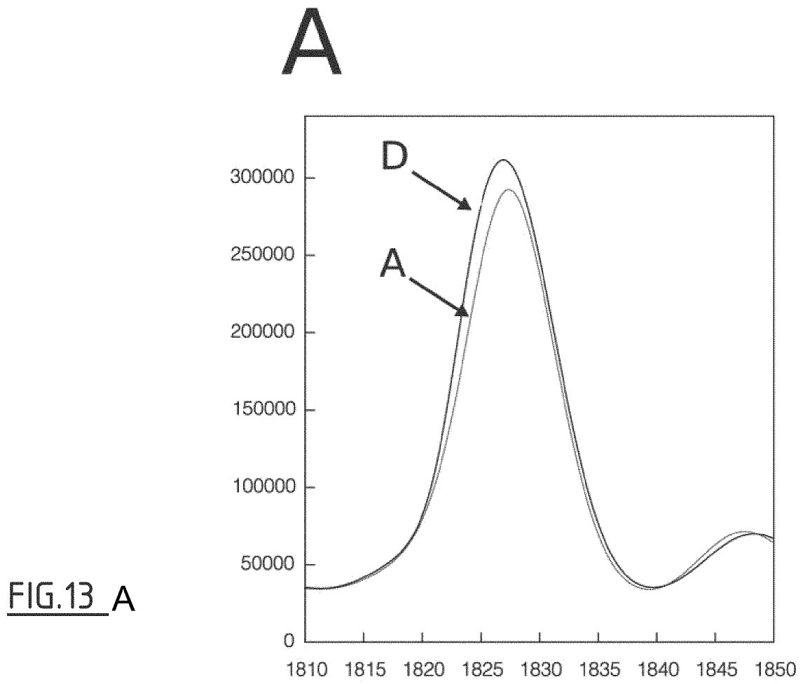

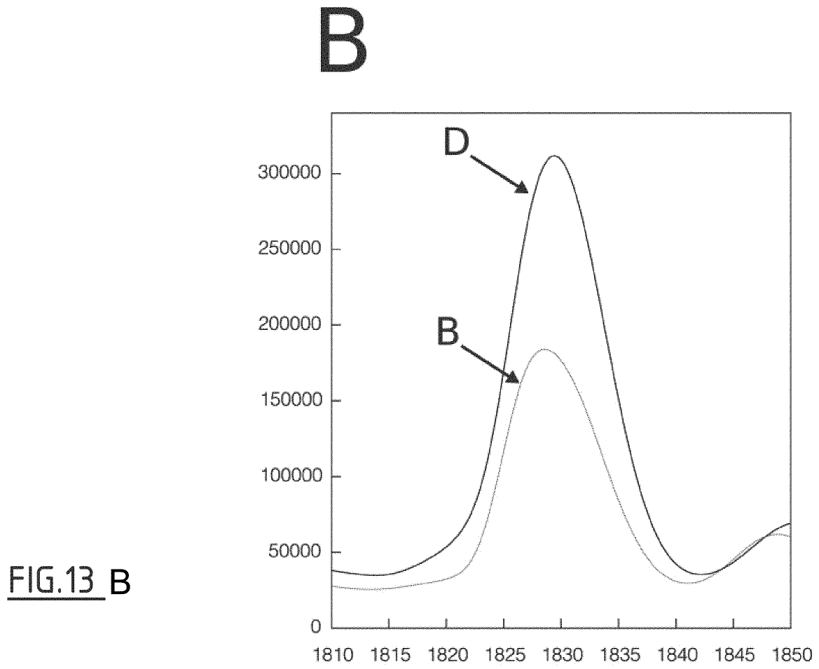

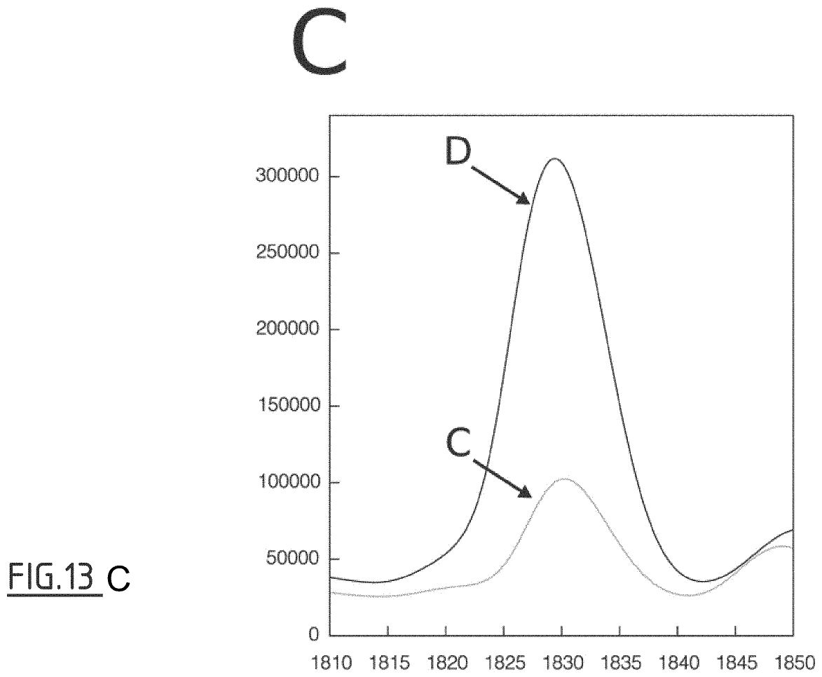

A second series of tests presented below relate to the aminopeptidases MjTET and PhTET4. An overlay of a fraction of the chromatograms obtained is shown in FIG. 13. In this case, it is observed that the aminopeptidase PhTET4, specific for glycine residues, is not capable of hydrolyzing peptides when it is used alone. There is thus a very clear overlay of the control chromatogram and that obtained after incubation with PhTET4. On the contrary, MjTET is capable of hydrolyzing several peptides present in the peptide mixture.

The remarkable result in this series of tests is linked to the fact that, when MjTET and PhTET4 are mixed in a composition, there is a significant increase in the hydrolysis of the peptides of the substrate.

Again, this unpredictable result shows how far it is possible to modulate the hydrolysis of various peptides specifically in a mixture using a characteristic TET aminopeptidase composition. The various results obtained to date, show that it is also possible to modulate the activity of the TET aminopeptidases as a function of the physicochemical conditions of the reaction medium. We therefore propose a process based on the exceptional properties of these enzymes.

### Composition PhTET1/PhTET2/PhTET3/Thermolysin

The whey used in these experiments comes from a cheese factory in Haute-Savoie. The whey was transported at 4° C. before being divided into 1 ml samples stored at −20° C.

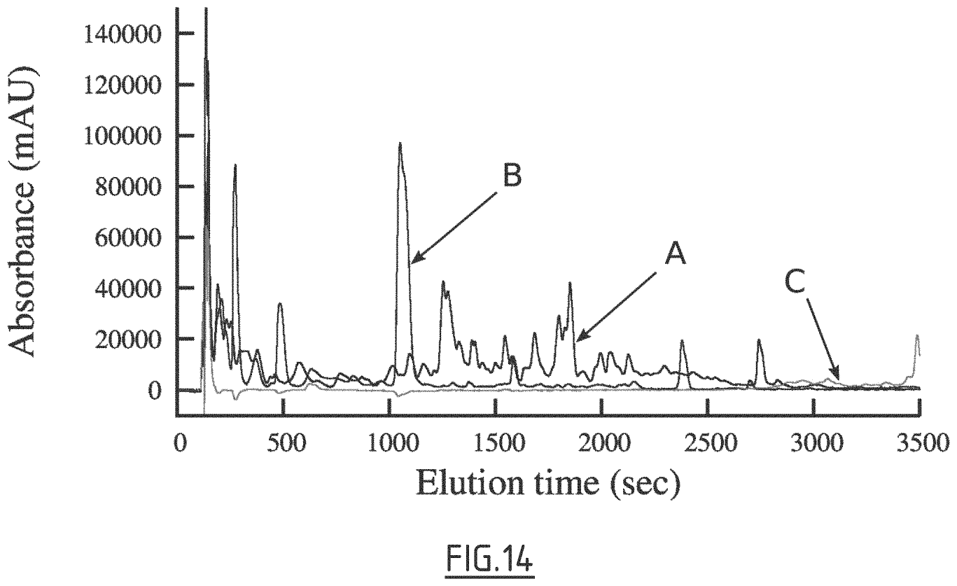

After incubation with thermolysin (FIG. 14), a large number of small peptides are present in the samples, the result of the hydrolysis of whey proteins by thermolysin.

It is remarkable to note that after incubation with thermolysin and TETs, the vast majority of these peptides have been degraded. We note the enrichment of some peptides which represent the degradation products linked to the activity of TET aminopeptidases.

Hydrolysis of a Specific Peptide within a Casein Hydrolyzate

### Composition PhTET3, MjTET and PhTET4 (33/33/33%)

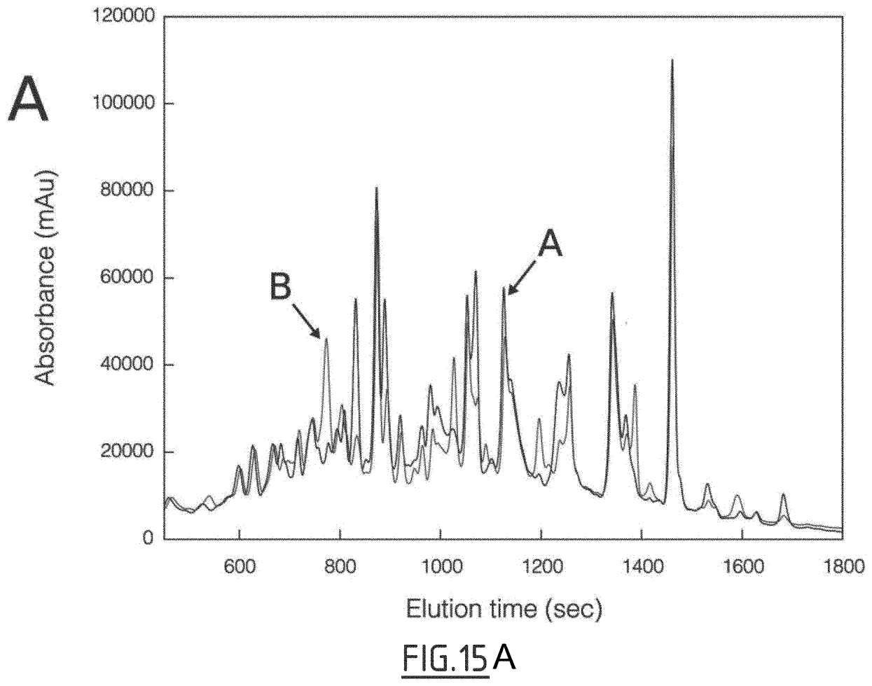

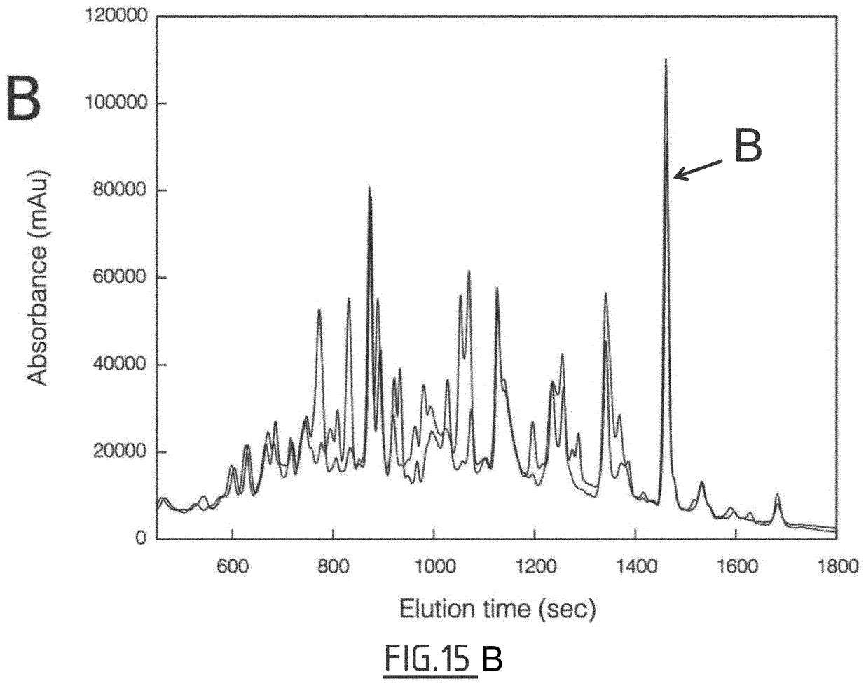

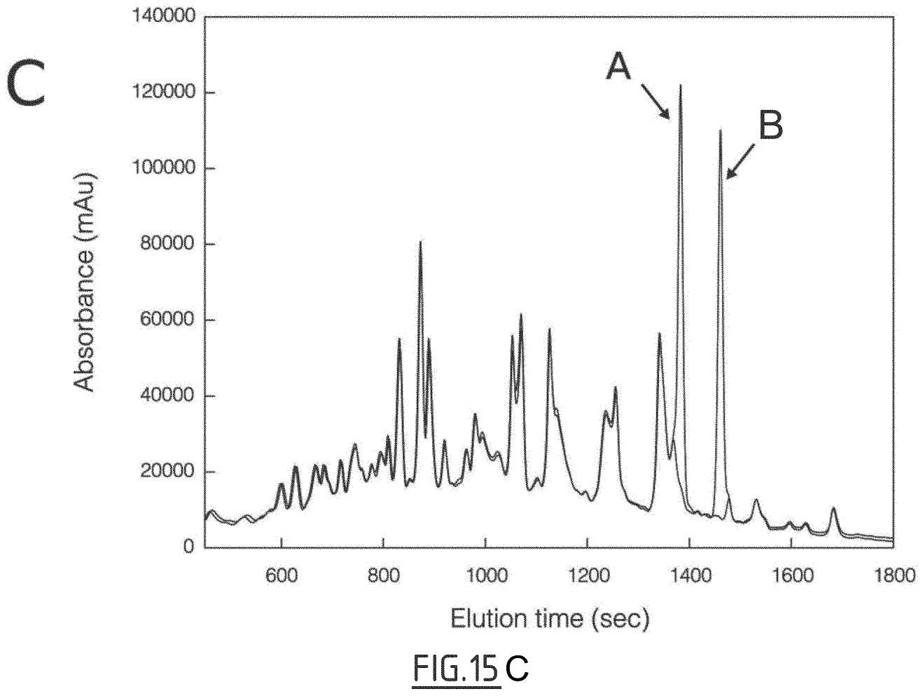

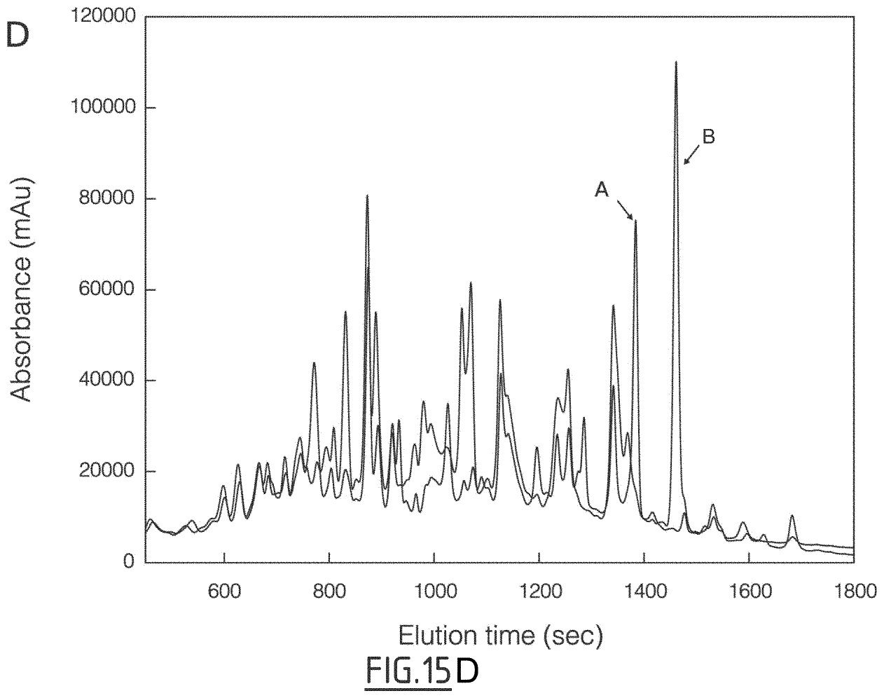

In this experiment, synthetic peptide 7 was incorporated into a mixture of complex peptide, a casein hydrolyzate (Sigma). After incubation with a composition of aminopeptidases PhTET3, MjTET and PhTET4, the reaction medium was analyzed by RP-HPLC. The results of the various experiments carried out are shown in FIG. 15. The peptide may be identified without ambiguity and its first stages of degradation could be observed. The complexity of the environment has made the analysis of short fragments more complex.

When the mixture of casein hydrolyzate and peptide 7 is incubated with the aminopeptidases PhTET3 or MjTET, it is observed that the peptide of interest is not very hydrolyzed (FIGS. 15A and B). As might be expected, however, a small number of peptides from the casein hydrolysis of the mixture are at least partly hydrolyzed by the TET aminopeptidases.

When the mixture is incubated in the presence of PhTET4, only the peptide of interest is hydrolysed by the aminopeptidase PhTET4 and, this, almost completely (FIG. 15C), the only peptide appearing being the peptide pep-1.

The intact peptide pep-0 of interest is also completely absent when the mixture of peptides is incubated in the presence of the composition of the three TET aminopeptidases (FIG. 15D). On the other hand, in the latter case, it may be noted that the peptide pep-1 has itself been hydrolyzed, since its concentration has decreased significantly compared to the experience with PhTET4 alone.

This experiment shows that the method makes it possible to precisely target a peptide in a mixture in order to modify or eliminate it.

### Specific Hydrolysis of Part of the Native Gluten Proteins

### Composition PhTET1/PhTET2/PhTET3 (33/33/33%)

Gluten is a mixture of various proteins classified into 2 main families, glutenins and gliadins. Some gliadins carry a peptide called “immunodominant” which causes an allergic reaction in people sensitive or intolerant to gluten; this syndrome is better known as Celiac disease.

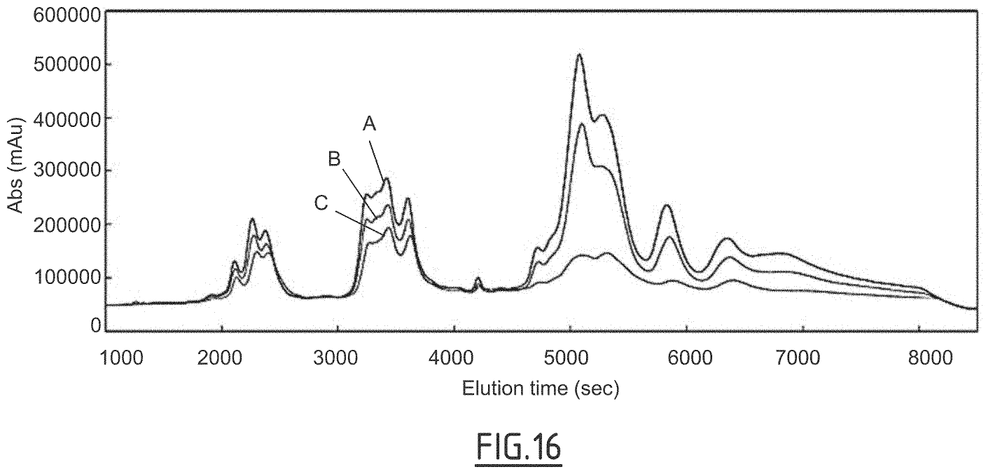

On the chromatograms shown in FIG. 16, it is observed that after incubation of a sample of total gluten solubilized in propanol with an equimolar quantity composition of the aminopeptidases PhTET1, PhTET2 and PhTET3, a significant decrease in the concentration is noted as gliadin in the sample.

In this case, the composition of the aminopeptidases PhTET1, PhTET2 and PhTET3 alone, i.e. without adding endopeptidase, made it possible to reduce the concentration of proteins carrying the immunodominant peptide in a sample of total gluten dissolved in a 50% solution of propanol.

### Composition PhTET1/PhTET2/PhTET3/Thermolysin

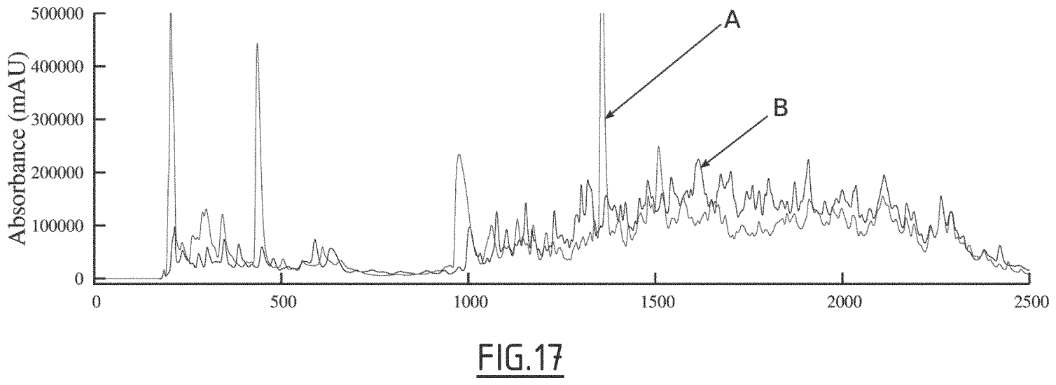

After incubation of the gluten with the thermolysin endoprotease, we note in FIG. 17 a large number of small peptides in the sample resulting from the very efficient hydrolysis of gluten proteins by the endoprotease. When the aminopeptidases PhTET1, PhTET2 and PhTET3, are integrated into the composition, there is a decrease in the vast majority of absorbance peaks, which translates to hydrolysis of all of these peaks by the aminopeptidases.

We also note an enrichment of some peaks which represent the degradation products of aminopeptidases.

### Example 2

### Results of Modification of Peptide Profile

### Material and Methods

### TET Aminopeptidase Activity Test on a Whey Protein Hydrolyzate

In order to test the hydrolysis activity of various combinations of TET aminopeptidases on the peptides present in the whey protein hydrolyzate, various mixtures of TET proteins at a total concentration of 50 μg/ml, are incubated with the hydrolyzate in a final volume of 100 μl. No cofactor is added to the reaction. The activity tests are carried out at pH=7.5 except those in which PhTET4 is present, and which are carried out at pH=9.5. The reaction medium is then incubated for 2 h at 60° C. with shaking (500 rpm). The tubes are then placed in ice to stop the hydrolysis reaction. Then, 80 μl of the reaction medium are added to 320 μl of a solution composed of 2% acetonitrile (ACN) and 0.1% trifluoroacetic acid (TFA). The samples are then centrifuged at 10,000 g for 10 min before being transferred to vials and their injection on an RP-HPLC column for analysis.

### Reverse Phase HPLC (RP-HPLC) Analysis

100 μl of each sample is injected onto a ZORBAX SB-300 C8 column (4.6 mm×150 mm) (Agilent®) connected to a Perkin Elmer® HPLC system. Phase A consists of 0.1% TFA and 2% ACN in water, phase B contains 0.1% TFA and 80% ACN in water. The adsorbed proteins are then eluted at 1 ml/min with a linear gradient 0-50% of phase B and are detected by measuring their absorbances at 280 nm for the studies on peptides, or 214 nm for the other studies. Protein peaks are identified and analyzed using TotalChrom software version 6.3.1 (Perkin Elmer®).

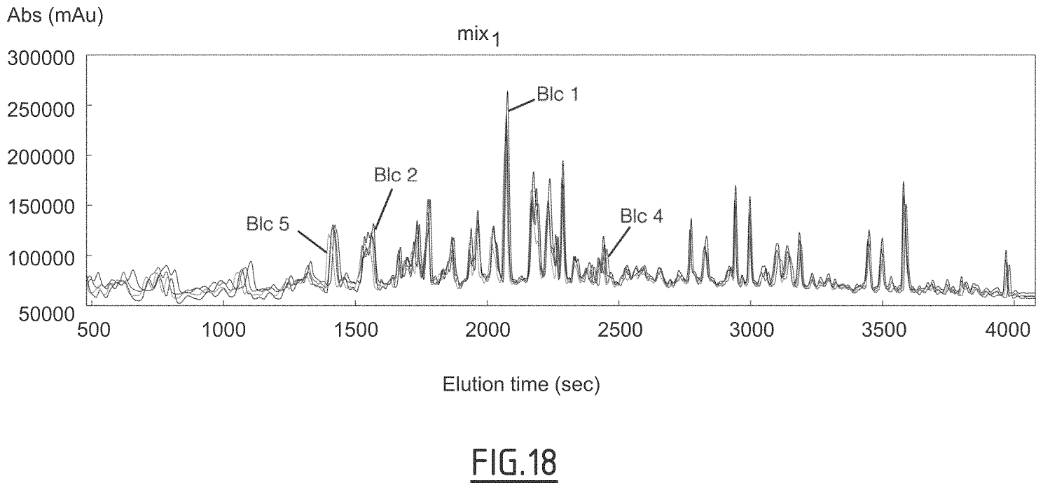

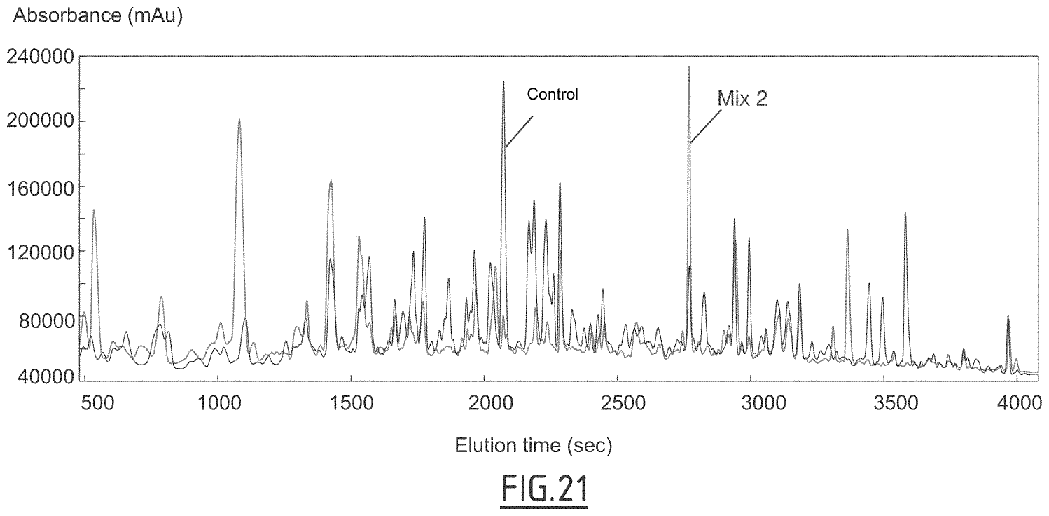

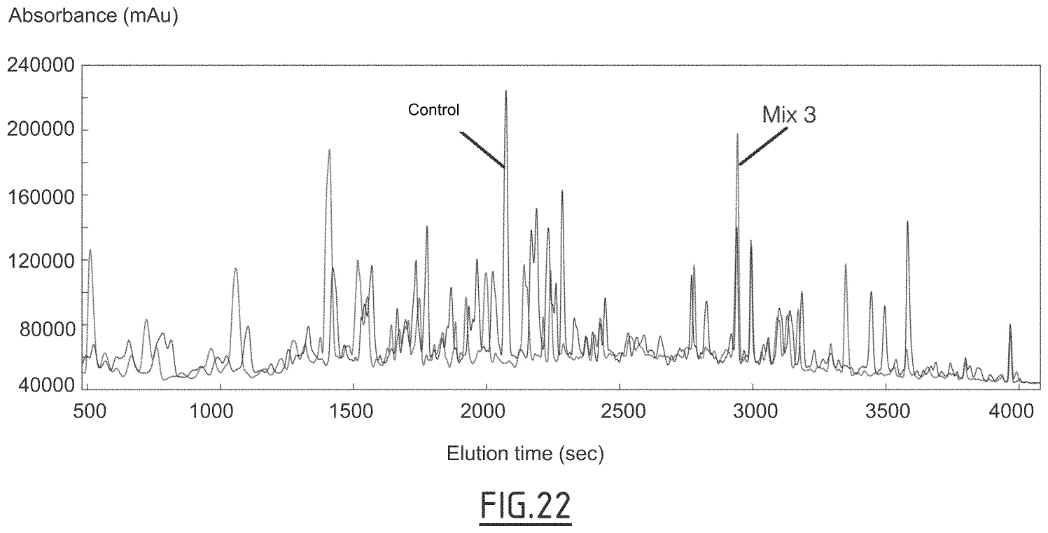

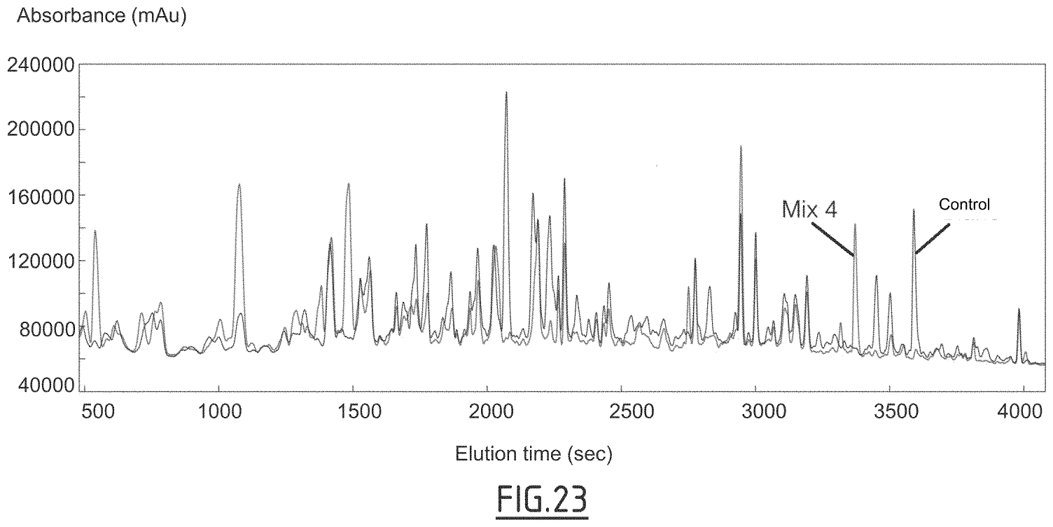

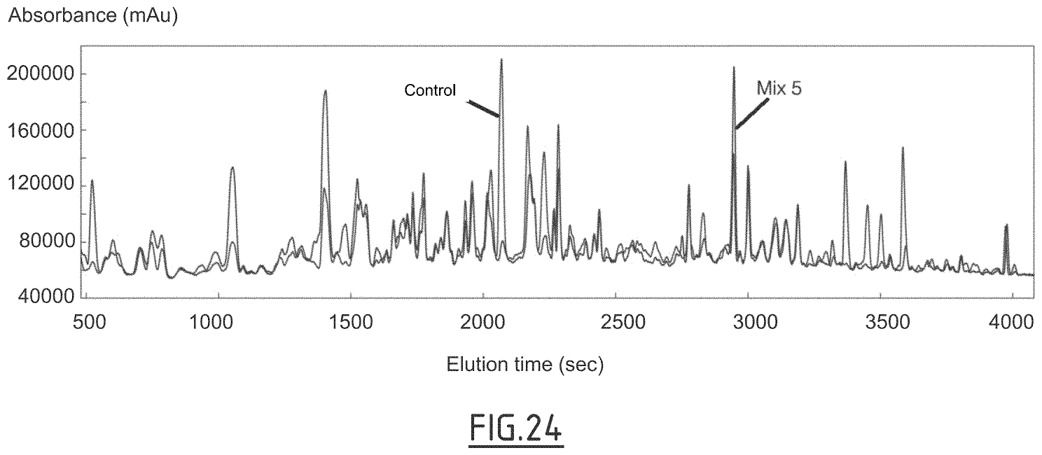

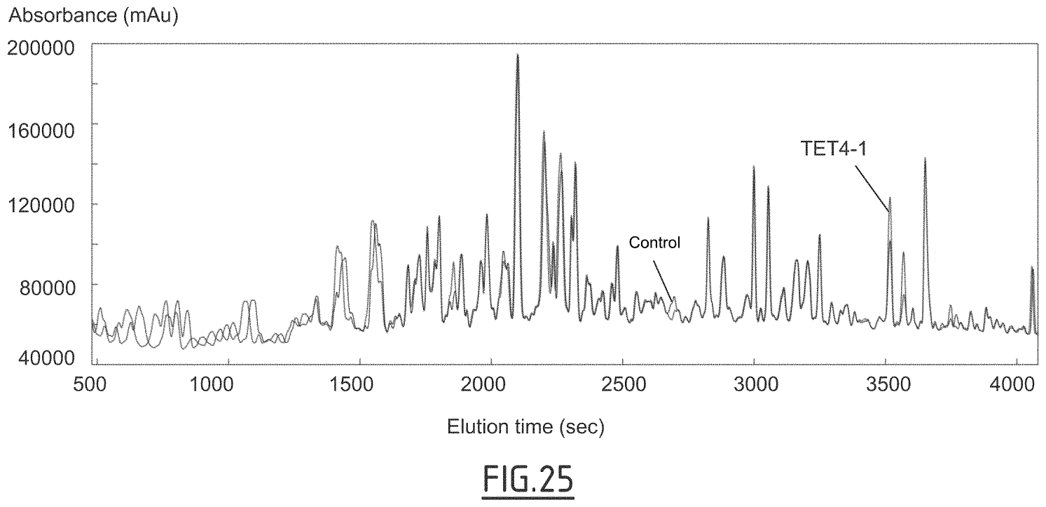

The “windows” of the chromatograms (FIGS. 18 to 28) are deliberately limited; only the elution gradient in itself is shown, i.e. a window between 480 sec and 4080 sec. The first minutes of the experiment correspond to the washing of the column after injection (0-480 sec), and the end of the washing after the gradient (4080-6000 sec).

FIGS. 18 to 28 show an overlay of the chromatograms resulting from RP-HPLC analyzes.

Below are described the various tests carried out with the mixtures (“mix” or “combinations”) defined above.

### Control of the Substrate Used

In order to ensure homogeneity and reproducibility in the analysis of the new whey protein hydrolyzate, the various “control” samples are overlaid and compared (FIG. 18).

Unfortunately, the mix 3 control sample is missing due to a technical problem.

It thus appears that the new hydrolyzate used, which in theory contains more diversity in the ends of the peptides, is completely homogeneous and stable, since the results of its analysis are perfectly reproducible.

### Hydrolysis of Peptides by the Different Mixes.

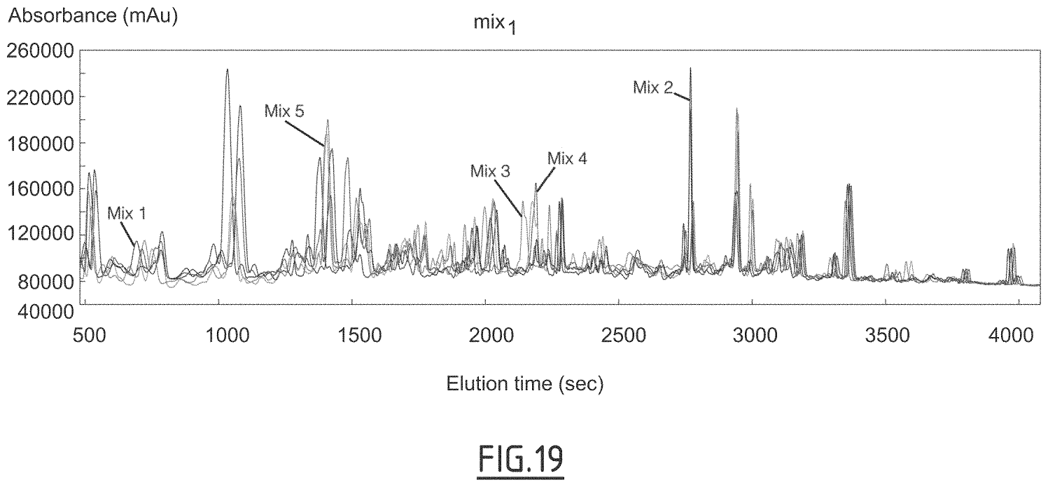

This whey protein hydrolyzate is incubated with various mixtures of TET. The overlaying of the chromatograms resulting from the RP-HPLC analysis of the samples after hydrolysis of the new whey protein hydrolyzate by different mixtures of TETs are shown in FIG. 19.

The overlay shown in FIG. 19 makes it possible to observe the significant variability of peptides obtained after hydrolysis by the different mixtures of TET. In the mixtures tested in this series of experiments, the majority of peptidases used are either PhTET2 or MjTET. Thus, it is very likely that the potential variability that may be obtained with the different TET mixes could be greater.

One thus obtains a “reshaping of the peptide profile” resulting from fine modifications of the peptides.

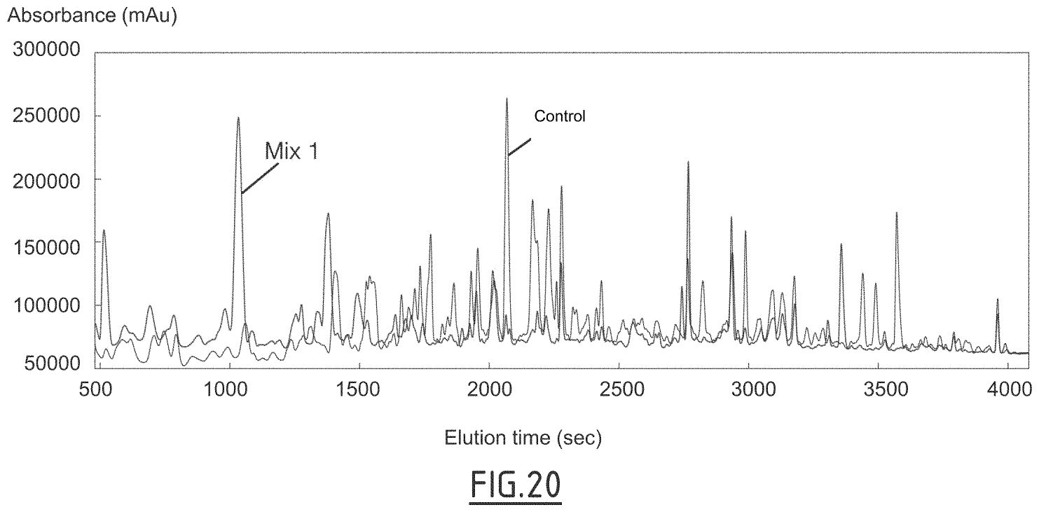

FIGS. 20 to 28 show different chromatograms obtained after the analysis in RP-HPLC of the reaction media after hydrolysis with the different mixtures of TET. The mix number and its composition are indicated in the caption.

The observation of these various mixtures clearly shows how the technology according to the invention makes it possible to “shape” a peptide profile.

It is possible to profoundly modify the nature of the peptides, without however completely degrading them: mix 1, 2. It is otherwise possible to modify the peptides in the medium more finely: mix 5, 6.

In order to better understand the advantage of mixing the different enzymes between them, several overlays are shown of the chromatograms obtained after hydrolysis by various mixtures or between the enzymes alone and as a mixture.

### Mix 1 and Mix 2

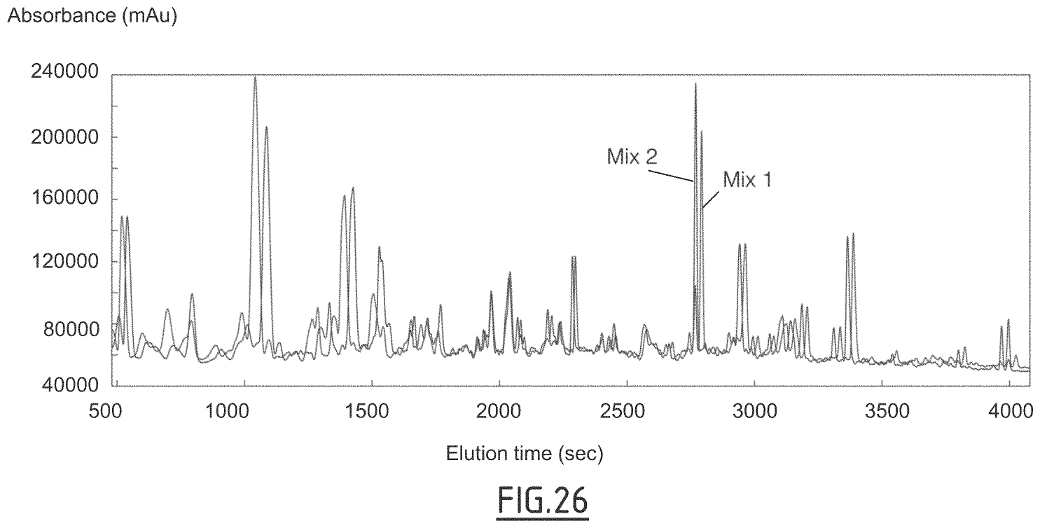

FIG. 26 represents one of the chromatograms resulting from the analysis in RP-HPLC of the hydrolysis of the peptides of the whey protein hydrolyzate by mixtures #1 and #2 of TET (Mix #1: 70% PhTET2, 15% PhTET3, 15% PhTET4; Mix #2: (70% PhTET2, 15% PhTET4, 15% MjTET).

There was a slight difference between the two runs.

The majority peptidase here is PhTET2, present in both mixes up to 70%. This majority presence of a peptidase explains the similarity that may be observed between the chromatograms. Although very similar, they are not identical. The only difference here is the presence in one case of PhTET3 and, in the other, of MjTET.

Studies on synthetic peptides have suggested that the peptides PhTET3 and MjTET have similar behavior in the peptide context. It appears here that there are still notable differences and that these two peptidases cannot be used to replace the other.

### Comparison TET2 VS MIX 1 and 2

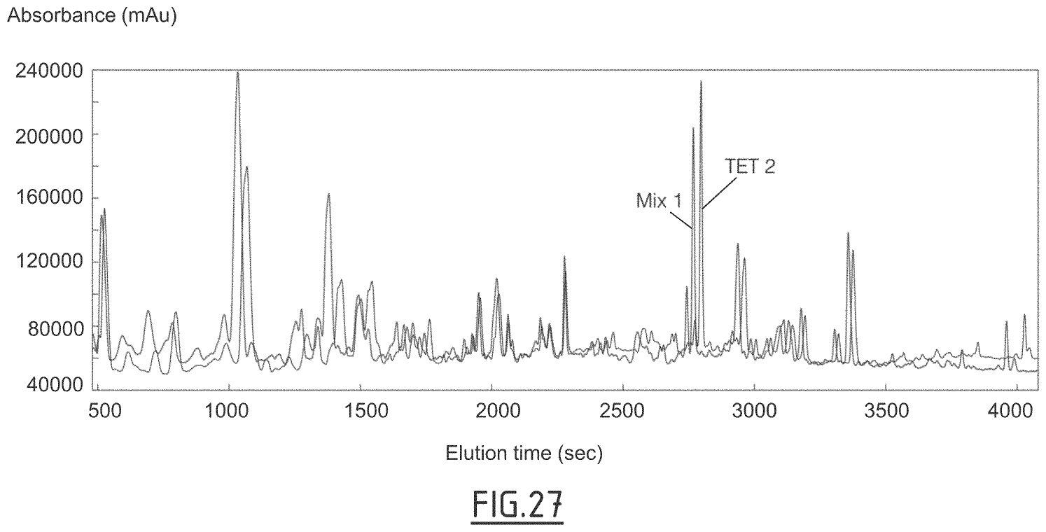

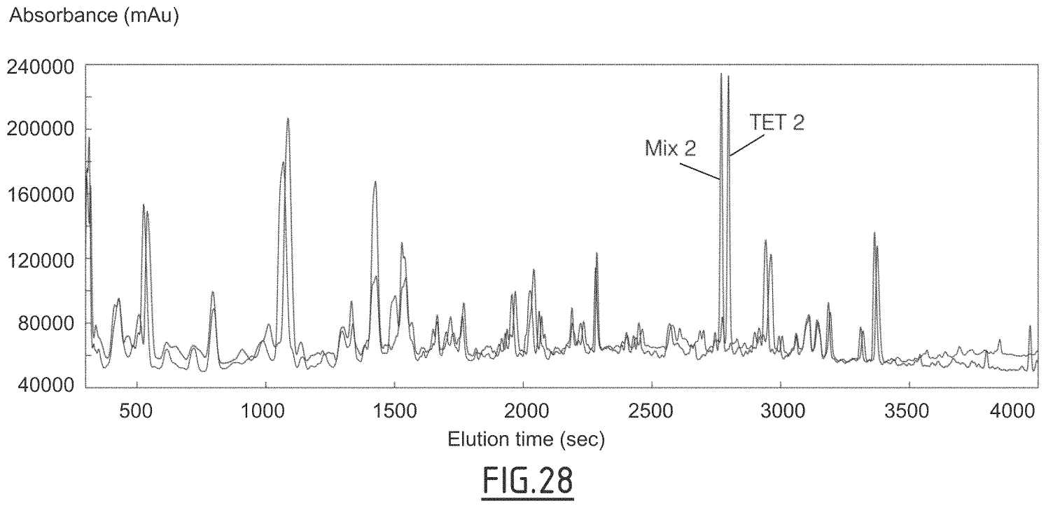

As said a few lines above, the majority peptidase, here PhTET2, strongly influences the general peptide profile. This is very clear in the present case, in particular according to FIGS. 27 and 28 with an overlay of the chromatograms resulting from the analysis in RP-HPLC of the hydrolysis of the peptides of the whey protein hydrolyzate by the mixtures #1 and #2 of TET (Mix #1: 70% PhTET2, 15% PhTET3, 15% PhTET4; Mix #2: (70% PhTET2, 15% PhTET4, 15% MjTET) each compared to PhTET2 respectively), since in both cases, the peptide profile after hydrolysis with peptidase alone is very close to that obtained after hydrolysis by the mixture of peptidases.

The few differences observed are due to “minority” peptidases. It is interesting to note that, in this case, these differences are relatively small compared to the modifications made by the majority peptidase.

### Example 3

### Crystallization of the Enzyme PhTET3

Crystals of the protein PhTET3 were obtained using the method of drops suspended on 24-well ComboPlate plates of the brand Greiner Bio-One. The crystals used for the experiments formed under a condition using the following mother liquor: 100 mM Tris-HCl, 100 mM NaCl, 100 mM (NH₄)SO₄, 43% of 2-Methyl-2,4-pentanediol and at pH=8. For crystallization, 1 ml of mother liquor is placed in the well of the crystallization plate, the drop is formed on silanized coverslips by mixing 1.5 μl of PhTET3 protein solution concentrated to 20 mg/ml and 1.5 μl of mother liquor.

### Cross-Linking of Crystals

In order to crosslink the PhTET3 crystals, a crosslinking solution is prepared from the mother liquor implemented with final 1% glutaraldehyde (v/v). Drops of 1 μl of crosslinking solution are deposited on silanized coverslips, the various crystals obtained earlier are then transferred into these drops. Crosslinking is obtained by incubation for 1 night; the crosslinked crystals are then harvested and placed in drops of the initial mother liquor while waiting to be used. The crosslinked crystal obtained has a larger dimension of at least 0.5 mm.

### Cross-Linked Enzyme Activity Tests

The objective being to be able to use these crosslinked enzyme crystals in industrial enzymatic processes, experiments were carried out in order to check whether the enzymes are still active once crosslinked.

In order to facilitate the observation of the activity of the crystals, a chromogenic substrate was used, in this case Lys-pNA. The crosslinked PhTET3 crystals were, therefore, incubated in a so-called activity buffer composed of 150 mM NaCl and 50 mM PIPES at pH=7.5 containing the Lys-pNA substrate at a concentration of 5 mM. This substrate was selected, in particular, because the enzyme PhTET3 has maximum activity against the amino acid lysine.

It may be observed that a drop of 1 μl of activity buffer in which a crosslinked crystal of PhTET3 has been incubated for 10 min at room temperature substantially has a diameter of several millimeters, typically 6-7 mm (observed on a drop of 1 μl of Lys-pNA substrate at 5 mM after incubation with a crosslinked crystal of pHTET3 for 10 min at room temperature). We may thus clearly observe the bright yellow color of the drop sign of a significant hydrolysis of the Lys-pNA substrate. This experiment shows that the enzyme PhTET3, once crystallized and crosslinked, is still active. It is a rare example of a large enzyme complex still active after crosslinking.

### Tests of Stability of Crosslinked Crystals

In order to be able to use these crystals in the processes mentioned, their mechanical strength and their physico-chemical stability were evaluated.

The crystals were subjected to 10 cycles of centrifugation-suspensions in a buffer solution containing 150 mM NaCl, 50 mM PIPES at pH=7.5 to 16,000 g without any loss of integrity or activity of the crystals being observed.

The same crystals were incubated at 90° C. for 1 h in the same buffer; again, no loss of integrity or activity of the crystals was observed after incubation.

They were also incubated in milli-Q distilled water for 7 days without loss of crystal integrity or activity. This last experiment is particularly interesting since it is not possible to observe such stability with the same non-crystallized enzyme.

### CONCLUSION

These results show that it is possible to produce CLECs which exhibit hydrolysis activity from TET enzymes. In addition, these crystals have quite remarkable properties of mechanical resistance and physicochemical stability. In an industrial context, these macroscopic crystals may easily be filtered after incubation with the substrate. They thus represent a strategy for immobilizing enzymes which is entirely realistic from an industrial and original point of view.

1. A composition comprising at least one first aminopeptidase and at least one second aminopeptidase, said first and second aminopeptidases are different from each other, said first and second aminopeptidases being isolated from extremophilic microorganisms, said first and second aminopeptidases being aminopeptidases from the family of tetrahedral aminopeptidases or TET aminopeptidases, said first aminopeptidase representing up to 40% by weight relative to the total weight of the composition, and, if said first and second aminopeptidases are different from PhTET2 and PhTET3, then said first aminopeptidase represents up to at 50% by weight relative to the total weight of the composition.

2. The composition according to claim 1, wherein said at least one first aminopeptidase and said at least one second aminopeptidase are chosen from aminopeptidases from the group consisting of: PhTET1, PhTET2, PhTET3, PhTET4 and MjTET.

3. The composition according to claim 2, in which said aminopeptidases comprise, consist essentially of, or consist of amino acid molecules of respective sequences SEQ ID NO: 1 to SEQ ID NO: 5, or proteins exhibiting aminopeptidase activity, said proteins comprising, consisting essentially of, or consisting of amino acid molecules whose sequences have at least 65% identity with one of the sequences SEQ ID NO: 1 to SEQ ID NO: 5.

4. The composition according to claim 1, wherein said first aminopeptidase represents up to 10% by weight relative to the total weight of the composition, in particular up to 5% by weight relative to the total weight of the composition.

5. The composition according to claim 1, wherein said first aminopeptidase represents 50% by weight relative to the total weight of the composition, provided that said first and second aminopeptidases are different from PhTET2 and PhTET3.

6. The composition according to claim 1, comprising at least a third aminopeptidase, said third aminopeptidase being an aminopeptidase from the family of tetrahedral aminopeptidases or TET aminopeptidases.

7. The composition according to claim 6, wherein said first, second and third aminopeptidases are in equimolar or substantially equimolar proportions.

8. The composition according to claim 1, further comprising an endopeptidase, in particular thermolysin, in particular thermolysin of sequence SEQ ID NO: 6.

9. The composition according to claim 1, wherein one or more aminopeptidases are in the form of crosslinked crystals.

10. A method for the modification of all or part of the polypeptide content of a substrate comprising peptides, polypeptides and/or proteins, said method comprising modifying the polypeptide content of a substrate comprising peptides, polypeptides and/or proteins by a composition according to claim 1.

11. The method according to claim 10, wherein the substrate comprises at least peptides, polypeptides and/or proteins of gluten and/or whey.

12. The method according to claim 10, wherein the substrate comprises at least one of the following proteins: gliadin, ß-lactoglobulin, α-lactalbumin, immunoglobulins, serum albumin and lactoferrin.

13. The method according to claim 10, wherein said aminopeptidases are used simultaneously, separately or spread over time.

14. A method for modifying all or part of the polypeptide content of a substrate comprising peptides, polypeptides and/or proteins, said method comprising a contacting step:
said substrate with
a composition according to claim 1,
said at least one first aminopeptidase and said at least one second aminopeptidase may be activated at a temperature above 80° C., and optionally comprising, prior to said contacting step, a step of denaturing the polypeptides of said substrate.

15. A food compound capable of being obtained by the method according to claim 14.

16. A food compound comprising at least one of the following proteins in modified form: gliadin, ß-lactoglobulin, α-lactalbumin, immunoglobulins, serum albumin and lactoferrin, said food compound further comprising a composition according to claim 1.


SEQUENCE LISTING (17 sequences)

SEQ ID NO: 1 - protein, 332 aa, Pyrococcus horikoshii
MMSMIEKLKKFTQIPGISGYEERIREEIIREIKDFADYKVDAIGNLIVELGEGEERILFMAHMDEIGLLITGITDEGKLRFRKVGGIDDRLLYGRHVNVVTEKGILDGVIGATPPHLSLERDKSVIPWYDLVIDIGAESKEEALELVKPLDFAVFKKHFSVLNGKYVSTRGLDDRFGVVALIEAIKDLVDHELEGKVIFAFTVQEEVGLKGAKFLANHYYPQYAFAIDSFACCSPLTGDVKLGKGPVIRAVDNSAIYSRDLARKVWSIAEKNGIEIQIGVTGGGTDASAFQDRSKTLALSVPIKYLHSEVETLHLNDLEKLVKLIEALAFEL

SEQ ID NO: 2 - protein, 353 aa, PYROCOCCUS HORIKOSHII
MEVRNMVDYELLKKVVEAPGVSGYEFLGIRDVVIEEIKDYVDEVKVDKLGNVIAHKKGEGPKVMIAAHMDQIGLMVTHIEKNGFLRVAPIGGVDPKTLIAQRFKVWIDKGKFIYGVGASVPPHIQKPEDRKKAPDWDQIFIDIGAESKEEAEDMGVKIGTVITWDGRLERLGKHRFVSIAFDDRIAVYTILEVAKQLKDAKADVYFVATVQEEVGLRGARTSAFGIEPDYGFAIDVTIAADIPGTPEHKQVTHLGKGTAIKIMDRSVICHPTIVRWLEELAKKHEIPYQLEILLGGGTDAGAIHLTKAGVPTGALSVPARYIHSNTEVVDERDVDATVELMTKALENIHELKI

SEQ ID NO: 3 - protein, 354 aa, PYROCOCCUS HORIKOSHII
MDLKGGESMVDWKLMQEIIEAPGVSGYEHLGIRDIVVDVLKEVADEVKVDKLGNVIAHFKGSSPRIMVAAHMDKIGVMVNHIDKDGYLHIVPIGGVLPETLVAQRIRFFTEKGERYGVVGVLPPHLRRGQEDKGSKIDWDQIVVDVGASSKEEAEEMGFRVGTVGEFAPNFTRLNEHRFATPYLDDRICLYAMIEAARQLGDHEADIYIVGSVQEEVGLRGARVASYAINPEVGIAMDVTFAKQPHDKGKIVPELGKGPVMDVGPNINPKLRAFADEVAKKYEIPLQVEPSPRPTGTDANVMQINKEGVATAVLSIPIRYMHSQVELADARDVDNTIKLAKALLEELKPMDFTP

SEQ ID NO: 4 - protein, 336 aa, PYROCOCCUS HORIKOSHII
MERIVKILREILEIPSPTGYTKEVMSYLEKFLKENEVNFYYTNKGALIAGNHPKPELVVIAHVDTLGAMVKEILPDGHLAFSRIGGLVLPTFEGEYCTIITRKGKKFRGTLLLRNPSAHVNREVGKKERKEENMYIRLDELVEKREDTEKLGIRPGDFIAFDPKFEYVNGFVKSHFLDDKASVAAILDLIIDMKDELEKYPVAFFFSPYEEVGHGGSAGYPPTTKELLVVDMGVVGEGVSGKETAVSIAAKDTTGPYDYDMTNRLIELAEENNIPYVVDVFPYYGSDGSAALRAGWDFRVALIGPGVHASHGMERTHVKGLLATKELIRAYIKWKG

SEQ ID NO: 5 - protein, 350 aa, METHANOCALDOCOCCUS JANNASCHII
MSVVEYLKKLSKLHGISGREDSVREFMKKELEKYCDSVEIDNFGNLIAKRGNKGKKIMIAAHMDEIGLMVKYIDDNGFLKFTKIGGIYDPTILNQKVVVHGSKGDLIGVLGSKPPHRMKEEEKTKIIKYEDMFIDIGAESREEAIEMGVNIGTWVSFLSEVYDLGKNRLTGKAFDDRVGCAVLLEVMKRLSEEDIDCQVYAVGTVQEEVGLKGARVSAFKINPDVAIALDVTIAGDHPGIKKEDAPVDLGKGPVVGIVDASGRGLIAHPKVLDMIKAVSEKYKIDVQWEVGEGGTTDATAIHLTREGIPTGVISVPARYIHTPVEVIDKRDLEKTVELVYNCIKEVNNFF

SEQ ID NO: 6 - protein, 551 aa, GEOBACILLUS STEAROTHERMOPHILUS
MKRKMKMKLVRFGLAAGLAAQVFFLPYNALASTEHVTWNQQFQTPQFISGDLLKVNGTSPEELVYQYVEKNENKFKFHENAKDTLQLKEKKNDNLGFTFMRFQQTYKGIPVFGAVVTAHVKDGTLTALSGTLIPNLDTKGSLKSGKKLSEKQARDIAEKDLVANVTKEVPEYEQGKDTEFVVYVNGDEASLAYVVNLNFLTPEPGNWLYIIDAVDGKILNKFNQLDAAKPGDVKSITGTSTVGVGRGVLGDQKNINTTYSTYYYLQDNTRGNGIFTYDAKYRTTLPGSLWADADNQFFASYDAPAVDAHYYAGVTYDYYKNVHNRLSYDGNNAAIRSSVHYSQGYNNAFWNGSQMVYGDGDGQTFIPLSGGIDVVAHELTHAVTDYTAGLIYQNESGAINEAISDIFGTLVEFYANKNPDWEIGEDVYTPGISGDSLRSMSDPAKYGDPDHYSKRYTGTQDNGGVHINSGIINKAAYLISQGGTHYGVSVVGIGRDKLGKIFYRALTQYLTPTSNFSQLRAAAVQSATDLYGSTSQEVASVKQAFDAVGVK

SEQ ID NO: 7 - protein, 7 aa, Artificial sequence
  Synthetic peptide
YTSWNSE

SEQ ID NO: 8 - protein, 15 aa, Artificial sequence
  synthetic peptide
EEDEKRRKYTSWNSE

SEQ ID NO: 9 - protein, 15 aa, Artificial sequence
  synthetic peptide
LMLLNENRYTSWNSE

SEQ ID NO: 10 - protein, 15 aa, Artificial sequence
  synthetic peptide
KRRKEEDEYTSWNSE

SEQ ID NO: 11 - protein, 15 aa, Artificial sequence
  synthetic peptide
GGGGNENRYTSWNSE

SEQ ID NO: 12 - protein, 15 aa, Artificial sequence
  synthetic peptide
YFLMNENRYTSWNSE

SEQ ID NO: 13 - protein, 15 aa, Artificial sequence
  synthetic peptide
EYNKAGRTELFQISV

SEQ ID NO: 14 - protein, 15 aa, Artificial sequence
  synthetic peptide
GISEQFKTNVLREYA

SEQ ID NO: 15 - protein, 15 aa, Artificial sequence
  synthetic peptide
FKYVGQNEISRATLE

SEQ ID NO: 16 - protein, 15 aa, Artificial sequence
  synthetic peptide
KLVREIYEQFNGTAS

SEQ ID NO: 17 - protein, 15 aa, Artificial sequence
  synthetic peptide
LNEGFTEKQSVARIY